(19) United States
(12) Patent Application Publication
(10) Pub. No.: US 2024/0145040 A1
(43) Pub. Date: May 2, 2024

(54) SYSTEMS AND METHODS FOR PARTICLE OF INTEREST DETECTION

(71) Applicant: Hyperspectral Corp., Alexandria, VA (US)

(72) Inventors: Euan F. Mowat, Concord, MA (US); Matthew Theurer, Alexandria, VA (US); Zachary Shaffer, Boston, MA (US)

(73) Assignee: Hyperspectral Corp., Alexandria, VA (US)

(21) Appl. No.: 18/496,650

(22) Filed: Oct. 27, 2023

Related U.S. Application Data
(60) Provisional application No. 63/381,269, filed on Oct. 27, 2022.

(51) Int. Cl. G16B 40/10; G01N 21/94; G16B 15/00

(52) U.S. Cl. CPC G16B 40/10; G01N 21/94; G16B 15/00

(57) ABSTRACT

An example method comprising: receiving a first set of intensity values based on a first set of intensity measurements for a set of wavelengths, the set of intensity measurements obtained by an apparatus configured to generate light and another light, detect the light that has passed through a portion of a sample, and measure intensity of the light by optical sensor, an angle separating the optical sensor and apparatus; applying a trained model to obtain a result; based on the result, determining a subset of wavelengths; receiving another set of intensity values, another set of intensity values being based on another set of intensity measurements for the set of wavelengths, the other set of intensity measurements obtained by the other light, detect the other light that has passed through another portion of the sample, and measure intensity by the optical sensor, another angle separating the optical sensor and the apparatus, the angles being different, applying another trained model to obtain another result, trained models being different from each other; applying the results to a trained multi-model to determine a positive or a negative pathogen detection for the pathogen in the sample, generating a notification and providing the notification.

### CROSS-REFERENCE TO RELATED APPLICATIONS

This application claims priority to U.S. Provisional Patent Application No. 63/381,269, filed on Oct. 27, 2022, and entitled “SYSTEMS AND METHODS FOR USING ARTIFICIAL INTELLIGENCE AND MACHINE LEARNING, IN CONJUNCTION WITH DYNAMIC LIGHT SCATTERING, USING AN INEXPENSIVE INCOHERENT BROAD SPECTRUM LIGHT SOURCE,” which is incorporated in its entirety herein by reference.

### FIELD OF THE INVENTION(S)

Embodiments of the present invention(s) are generally related to detecting particles of interest utilizing incoherent light sources.

### BACKGROUND

Detecting pathogens, chemicals, and/or other substances is a difficult process and often leads to inaccurate detections (e.g., false positives and false negatives) and is problematic to do at scale. There is an increasing need for such detection, however, in that undetected substances can cause disease, failures, or unintended consequences.

For example, undetected foodborne illnesses may be caused by consuming food or beverages that are contaminated by pathogens such as bacteria, toxins produced by bacteria, viruses, parasites, chemicals, foreign material (e.g., metal shavings), and/or the like. The United States Food and Drug Administration (U.S. FDA) estimates that there are approximately 48 million cases of foodborne illness each year in the United States. The U.S. FDA further estimates that 1 in 6 Americans are affected by foodborne illnesses, resulting in 128,000 hospitalizations and 3,000 deaths per year.

Food or beverages (collectively, food) may be contaminated during any stage in the supply chain (e.g., in the field, while undergoing processing at food production or processing facilities (collectively, food processing facilities), or during shipping or transport). However, the contamination may not be discovered until after people are sickened from consuming the food. Unfortunately, government agencies, such as the U.S. FDA, often declare an outbreak of a foodborne illness and issue recalls of the food suspected of causing the outbreak only after a number of people are sickened.

In addition to the deleterious effects on individual health, there are economic costs to recalls. For example, a food producer or processor (collectively, a food processor) may voluntarily or be required to recall numerous lots of food or entire production runs. Such recalls may sicken many and may tarnish the brand of the food processor, leading to consumer distrust, reduced sales, and high costs for product recalls, legal defense, and damage control.

Foodborne pathogens is just one example. Processes for detecting the existence of chemicals and other substances is required but current methods may not provide fast and accurate solutions.

### SUMMARY

A example non-transitory computer-readable medium comprising executable instructions, the executable instructions being executable by one or more processors to perform a method, the method comprising: receiving a first set of intensity values, the first set of intensity values being based on a first set of intensity measurements for a set of wavelengths, the first set of intensity measurements obtained by an apparatus configured to generate first light, detect the first light that has passed through at least a first portion of a sample, and measure intensity of the first light by at least one optical sensor, a first angle separating the at least one optical sensor and the apparatus configured to generate light, applying a first trained model to the first set of intensity values to obtain a first result, based on the first result, determining a first subset of wavelengths of the first set of wavelengths, receiving a second set of intensity values, the second set of intensity values being based on a second set of intensity measurements for the set of wavelengths, the second set of intensity measurements obtained by the apparatus configured to generate second light, detect the second light that has passed through at least a second portion of the sample, and measure intensity of the second light by the at least one optical sensor, a second angle separating the at least one optical sensor and the apparatus configured to generate light, the first angle being different than the second angle, applying a second trained model to the second set of intensity values to obtain a second result, the first and second trained model being different from each other, applying the first and second result to a trained multi-model to determine either a positive pathogen detection or a negative pathogen detection for the pathogen in the sample, generating a pathogen detection notification that indicates either the positive pathogen detection or the negative pathogen detection for the pathogen in the sample; and providing the pathogen detection notification.

In one example, the example method further comprising: based on the output from the trained multi-model, determining a confidence level of the positive pathogen detection or the negative pathogen detection of the pathogen in the sample. In some embodiments, the method further comprising based on the output from the trained multi-model, determining a distribution of pathogen particle size in the sample. In various embodiments, the method further comprising based on the output from the trained multi-model, determining a concentration of the pathogen in the sample. In one example, the apparatus includes at least one incoherent light source. In some embodiments, the apparatus includes a plurality of light emitting diodes (LEDs) and wherein the first angle separating at least one optical sensor and the apparatus utilizes one of the plurality of LEDs and the second angle separating at least one optical sensor and the apparatus utilizes another of the plurality of LEDs. In another example, the at least one optical sensor includes at least two optical sensors and wherein the first angle separating at least one optical sensor and the apparatus utilizes a first optical sensor and the second angle separating at least one optical sensor and the apparatus is achieve utilizes a second optical sensor. In one example, the first angle separating the at least one optical sensor and the apparatus configured to generate light is substantially 180 degrees.

In one example, the first angle and the second angle are substantially the same and wherein the first set of intensity measurements is obtained by positioning the sample in a first position and the second set of intensity measurements is obtained by positioning the sample in a second position, the first position being different from the second position.

In some embodiments, the example method further comprising: receiving a third set of intensity values, the third set of values based on a third set of intensity measurements for the set of wavelengths, the third set of intensity measurements obtained by the apparatus configured to generate light, detect the light that has passed through at least the portion of the sample, and measure intensity of light at least one optical sensor, a third angle separating the at least one optical sensor and the apparatus configured to generate light, and applying a third trained model to the third set of intensity values to obtain a third result, wherein the determining either the positive pathogen detection or the negative pathogen detection for the pathogen in the sample is based on the output of the multi-model from the input of the first, second, and third results. In one example, at least some training samples of the set of training samples correspond to a particular food processing facility, a region that includes multiple food processing facilities, or one or more classes of food processing facilities. In another example, the result indicates the positive foodborne pathogen detection if the result meets or exceeds a threshold. In various embodiments, the set of wavelengths of light includes wavelengths of light in ultraviolet, visible, and infrared spectrums. In one example, the second set of intensity values is based on scattered radiation from the source, the second trained model being a trained to analyze scattered radiation and the first trained model being trained to analyze absorbed radiation. In one example, the at least one optical sensor comprises a first optical sensor that measures the intensity of the first light and a second optical sensor that measures the intensity of the second light. In some embodiments, the first trained model includes a first decision tree and the second trained model includes a second decision tree.

An example system comprising at least one processor and memory containing executable instructions, the executable instructions being executable by the at least one processor to: receive a first set of intensity values, the first set of intensity values being based on a first set of intensity measurements for a set of wavelengths, the first set of intensity measurements obtained by an apparatus configured to generate first light, detect the first light that has passed through at least a first portion of a sample, and measure intensity of the first light by at least one optical sensor, a first angle separating the at least one optical sensor and the apparatus configured to generate light, apply a first trained model to the first set of intensity values to obtain a first result, based on the first result, determine a first subset of wavelengths of the first set of wavelengths, receive a second set of intensity values, the second set of intensity values being based on a second set of intensity measurements for the set of wavelengths, the second set of intensity measurements obtained by the apparatus configured to generate second light, detect the second light that has passed through at least a second portion of the sample, and measure intensity of the second light by the at least one optical sensor, a second angle separating the at least one optical sensor and the apparatus configured to generate light, the first angle being different than the second angle, apply a second trained model to the second set of intensity values to obtain a second result, the first and second trained model being different from each other, applying the first and second result to a trained multi-model to determine either a positive pathogen detection or a negative pathogen detection for the pathogen in the sample, generate a pathogen detection notification that indicates either the positive pathogen detection or the negative pathogen detection for the pathogen in the sample, and provide the pathogen detection notification.

In one example, the executable instructions being executable by the at least one processor to determine a confidence level of the positive pathogen detection or the negative pathogen detection of the pathogen in the sample, based on the output from the trained multi-model. In another example, the executable instructions being executable by the at least one processor to generate the second set of values based on the first set of values include executable instructions being executable by the at least one processor to determine a distribution of pathogen particle size in the sample based on the output from the trained multi-model. An example system, the executable instructions being executable by the at least one processor to determine a concentration of the pathogen in the sample based on the output from the trained multi-model. In one example, the apparatus includes at least one incoherent light source. In another example, the apparatus includes a plurality of light emitting diodes (LEDs) and wherein the first angle separating at least one optical sensor and the apparatus utilizes one of the plurality of LEDs and the second angle separating at least one optical sensor and the apparatus utilizes another of the plurality of LEDs. In some embodiments, the at least one optical sensor includes at least two optical sensors and wherein the first angle separating at least one optical sensor and the apparatus utilizes a first optical sensor and the second angle separating at least one optical sensor and the apparatus is achieve utilizes a second optical sensor. In one example, the first angle separating the at least one optical sensor and the apparatus configured to generate light is substantially 180 degrees. In another example, the first angle and the second angle are substantially the same and wherein the first set of intensity measurements is obtained by positioning the sample in a first position and the second set of intensity measurements is obtained by positioning the sample in a second position, the first position being different from the second position.

In one example, the executable instructions being executable by the at least one processor to: receive a third set of intensity values, the third set of values based on a third set of intensity measurements for the set of wavelengths, the third set of intensity measurements obtained by the apparatus configured to generate light, detect the light that has passed through at least the portion of the sample, and measure intensity of light at least one optical sensor, a third angle separating the at least one optical sensor and the apparatus configured to generate light, and apply a third trained model to the third set of intensity values to obtain a third result, wherein the determining either the positive pathogen detection or the negative pathogen detection for the pathogen in the sample is based on the output of the multi-model from the input of the first, second, and third results. In one example, at least some training samples of the set of training samples correspond to a particular food processing facility, a region that includes multiple food processing facilities, or one or more classes of food processing facilities. In another example, the result indicates the positive foodborne pathogen detection if the result meets or exceeds a threshold. In yet another example, the set of wavelengths of light includes wavelengths of light in ultraviolet, visible, and infrared spectrums.

An example method comprising: receiving a first set of intensity values, the first set of intensity values being based on a first set of intensity measurements for a set of wavelengths, the first set of intensity measurements obtained by an apparatus configured to generate first light, detect the first light that has passed through at least a first portion of a sample, and measure intensity of the first light by at least one optical sensor, a first angle separating the at least one optical sensor and the apparatus configured to generate light, applying a first trained model to the first set of intensity values to obtain a first result, based on the first result, determining a first subset of wavelengths of the first set of wavelengths, receiving a second set of intensity values, the second set of intensity values being based on a second set of intensity measurements for the set of wavelengths, the second set of intensity measurements obtained by the apparatus configured to generate second light, detect the second light that has passed through at least a second portion of the sample, and measure intensity of the second light by the at least one optical sensor, a second angle separating the at least one optical sensor and the apparatus configured to generate light, the first angle being different than the second angle, applying a second trained model to the second set of intensity values to obtain a second result, the first and second trained model being different from each other, applying the first and second result to a trained multi-model to determine either a positive pathogen detection or a negative pathogen detection for the pathogen in the sample, generating a pathogen detection notification that indicates either the positive pathogen detection or the negative pathogen detection for the pathogen in the sample, and providing the pathogen detection notification.

### BRIEF DESCRIPTION OF THE DRAWINGS

FIG. 1 depicts an example particle detection environment in some embodiments.

FIG. 2 depicts an example particle detection environment with configurable angles of incidence detection according to some embodiments.

FIG. 3 is a block diagram of components of a pathogen detection system in some embodiments.

FIG. 4 is a flowchart showing a method for detecting pathogens in some embodiments.

FIG. 5A is a simplified diagram of a two-dimensional array for pathogen detection according to some embodiments.

FIG. 5B is a diagram of a two-dimensional array with wavelengths of interest according to some embodiments.

FIG. 5C depicts a method for detecting pathogens using incoherent light sources according to some embodiments.

FIG. 6A depicts a configuration of a spectral acquisition system where a light source is orthogonal to a sensor according to some embodiments.

FIG. 6B depicts a configuration of a spectral acquisition system where the light source is repositioned according to some embodiments.

FIG. 6C depicts a configuration of a spectral acquisition system where multiple light sources are utilized to acquire spectral measurements according to some embodiments.

FIG. 6D depicts a configuration of a spectral acquisition system with multiple light-emitting diodes (LEDs) according to some embodiments.

FIG. 7A depicts a configuration of a spectral acquisition system where a sensor is repositioned according to some embodiments.

FIG. 7B depicts a configuration of a spectral acquisition system where multiple sensors are utilized to acquire spectral measurements according to some embodiments.

FIG. 8 depicts a configuration of a spectral acquisition system where the sample is repositioned according to some embodiments.

FIG. 9 is a flowchart showing a method for training sets of decision trees for detecting pathogens in some embodiments.

FIG. 10 depicts a high-level diagram of the multi-dimensional array and high-level models according to some embodiments.

FIG. 11 depicts a block diagram of an example digital device in some embodiments.

Throughout the drawings, reference numerals will be understood to refer to parts, components, and structures.

### DETAILED DESCRIPTION

In various embodiments, systems, and methods discussed herein may leverage both absorption and scattered radiation to assist in detection of particles of interest. In one example, light from one or more light sources that pass through (directly through and at an angle relative to one or more receivers) a source (e.g., cuvette) may be measured (e.g., absorption, reflection, and/or transmission) and analyzed through separately trained models (e.g., decision tree models). The output of those models may pass through a multi-modal to assist in determining the particle(s) of interest. This process may be used to accurately detect pathogens, chemicals, food substances, proteins, and/or the like.

In some embodiments, systems discussed herein may utilize inexpensive incoherent light sources with broad spectrum. The angle of incidence of the incoherent light source, relative to the optical sensor(s), may be configurable to allow measurements of absorbance, reflectance, and, due to the configurable angle of incidence, measurements of dynamic light scattering. The reflection, absorption, and transmission of each wavelength may or may not contain information that is pertinent to the identification of a given particle. Furthermore, if multiple scans are performed (e.g., perhaps thousands of scans), the information captured by a single wavelength may vary over time (i.e., from scan to scan). Such a configuration may also capture time-based data that results from the Brownian motion of particles within the medium.

The systems may utilize light intensity measuring apparatuses, which may be or include spectrometers or spectrophotometers, to scan water used or produced by food processing apparatuses. The light intensity measuring apparatuses may transmit the spectrometer scans to a particle detection system that utilizes machine learning (ML) and/or artificial intelligence (AI) models to detect evidence of particles of interest in the spectrometer scans. The particle detection system may provide results to devices and users to assist in quality control, pathogen detection, and/or the like. For example, in the event of a positive detection of a foodborne pathogen, personnel may stop food processing and start remedial measures, such as cleaning food processing equipment, discarding contaminated food, and/or performing additional testing or detection.

Such early detection of foodborne pathogens allows food processors to identify contaminated food prior to shipping the food out to wholesalers, distributors, retailers, and/or consumers. This early detection may save food processors the costs of recalling food, which may be significant. In addition, early detection may prevent or reduce the occurrence of foodborne illness outbreaks, which may prevent or reduce illnesses, hospitalizations, and deaths.

In various embodiments, the systems and methods described herein are applicable to detect a wide variety of particles. Returning to the foodborne pathogen example, the some embodiments may be able to detect one or more of norovirus, salmonella (non-typhoidal), Clostridium perfringens, campylobacter, Staphylococcus aureus, Toxoplasma gondii, Escherichia coli (E. coli), Clostridium botulinum, cryptosporidium, Cyclospora, hepatitis A virus, shigella, Yersinia, Listeria monocytogenes (listeria), among many others. Similarly, systems and methods described herein may be capable to detect various chemicals used in manufacturing facilities (e.g., for semiconductor fabrication, chemical creation, chemicals in vapor or the atmosphere, or the like).

The particle detection systems may train one or more ML and/or AI models for each particle of interest. Upon receiving spectral data from light intensity measuring apparatuses, the particle detection systems may apply the trained machine learning and/or AI models to the spectral data. In this way, the particle detection systems may be able to detect multiple particles of interest from spectral data of a single sample. One advantage of some embodiments of the systems and methods described herein is that they may decrease the Limit of Detection (LOD) from the Classical Limit of Detection (cLOD), which is limited by physics, to the machine learning limit of detection (mlLOD) that may be one to two orders of magnitude lower than the cLOD.

Such systems may be located and utilized in many different locations and at many different points of the process. Food processors may widely deploy the spectral acquisition system at food processing facilities to detect foodborne pathogens in their food processing. The particle detection systems and associated methods described herein, because they provide more accurate results more quickly and economically than other systems and methods, are broadly applicable to any location where food is processed, such as farms, food processing facilities, packaging facilities, distributors, restaurants, grocery stores, homes, and other locations. Accordingly, the foodborne pathogen detection systems and associated methods described herein may provide significant benefits to farmers, food processors, distributors, restaurant operators, grocery store operators, households, consumers, and others (e.g., any entity in the farm-to-fork supply chain).

The particle detection systems and associated methods, due to the ability to perform rapid and continuous testing of foods, also allow for food processors to quarantine food that may be contaminated by foodborne pathogens prior to shipping out such food. For example, a food processor, upon detection of a foodborne pathogen during a particular food processing run, may be able to quarantine food processed during that run or food processed after the last “clean” test prior to shipping out that food. The food processor may then test the food (e.g., using laboratory tests) to confirm the presence of foodborne pathogens. The food processor may also be able to clean food processing equipment and/or parts of the food processing facility to prevent or reduce contamination of further food. The food processor may then retest food processing byproducts and/or equipment for contamination. As a result, the food processor may confirm that the machinery and/or byproducts are “clean” (e.g., without detected foodborne pathogens) before returning to food processing.

Accordingly, users may be able to reduce economic costs associated with foodborne illness outbreaks. Furthermore, effects on individual health and/or public health may be avoided or reduced by the deployment of the particle detection systems and associated methods described herein.

The particle detection systems and associated methods described herein may also aid manufacturers, processors, and food processors in complying with safety laws and regulations, such as those promulgated by government agencies.

In another example, the particle detection systems and associated methods may also be utilized to detect pathogens and/or contaminants that may affect water quality. Accordingly, the particle detection systems and associated methods described herein may also aid community water systems and/or other water suppliers with complying with water quality standards, such as those promulgated by government agencies such as the U.S. Environmental Protection Agency.

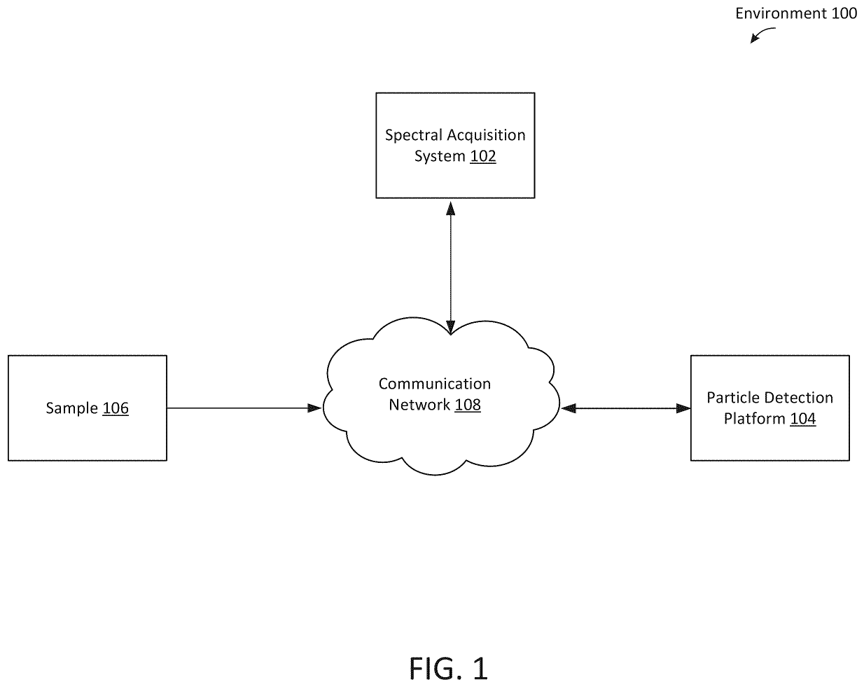

FIG. 1 depicts an example particle detection environment 100 in some embodiments. The particle detection environment 100 includes a sample 106 (referred to herein, for example, as a pathogen sample 106 or as pathogen samples 106), a spectral acquisition system 102, and a particle detection platform 104.

The sample 106 may be or include any device that samples the product or byproduct of machine and/or apparatus that processes such as a food processes or processes that facilitates processing food for human or animal consumption. For example, a pathogen sample 106 may be a sample of water from a washing machine that washes fruits and vegetables such as leafy greens, apples, carrots. In another example, the pathogen sample 106 may be a sample from a commercial spinner that dries washed lettuce and other vegetables, which produces water to be drained away.

In some embodiments, the spectral acquisition system 102 includes a light source (e.g., a source that generates electromagnetic radiation) and an optical sensor. In some embodiments, the light source may be an incoherent light source. An incoherent light source outputs electromagnetic energy with a broad spectrum, where light waves have random phases and are not synchronized. Although the term “light source” and “light” are referred to herein, it will be appreciated that the light being generated by the light source may be any electromagnetic radiation at different wavelength(s) and/or ranges of wavelength(s). As such, the light produced by the light source may be electromagnetic radiation may be in different nonvisible ranges.

The optical sensor may be an image capture device, such as a complementary metal-oxide semiconductor (CMOS) image sensor or a charge-couple device (CCD). In various embodiments, the spectral acquisition system 102 includes a spectrometer. The spectrometer may separate spectral components of the light source.

The separated spectral components may be directed toward the sample 106 and detected by the optical sensor. The optical sensor may then transmit a set of intensity values based on the measured intensities for the set of wavelengths of the light to the particle detection platform 104. The set of intensity values may be the measured intensities, or they may be other values based on the measured intensities, such as absorbance, transmittance values and/or scattering values.

In one example, the spectral acquisition system 102 includes multiple light sources and/or multiple optical sensors positioned in different locations to vary the angle of incidence of the light source relative to the optical sensor. The angle of incidence may be able to allow for measurements of light absorbance, reflectance, and measurements of dynamic light scattering. The angle of incidence may be configurable in many different ways.

For example, the spectral acquisition system 102 includes one light source that may be repositioned (e.g., moved) from one position (e.g., directly across from the optical sensor that measures wavelengths) to a second position (e.g., at a different position where the optical sensor may receive light reflected or at an angle from the light relative to the sample 106). In one example, the light source may transmit light through the sample 106 to the optical sensor (e.g., which may measure absorption and transmission). The light source may be moved to a second position and transmit light a second time through the sample. Light that is reflected (e.g., scattering radiation) may be detected by the optical sensor. In some embodiments, the light source may transmit as the light source is moved to the new position(s) (e.g., the light source continues to transmit light and does not deactivate and re-transmit light at different positions).

In some embodiments, there are multiple light sources that transmit serially (e.g., one at a time) or at the same time (e.g., simultaneously or near simultaneously) without moving the light sources.

In another example, the angle of incidence may be configurable by repositioning the optical sensor between an initial scan and subsequent scans. For example, the optical sensor may be in a first position and receive light transmitted from the light source through the sample 106 to the optical sensor. The optical sensor may then be moved to a second position, different from the first position, and the light source may, again, transmit light to the sample 106 and the optical sensor may receive light reflected or scattered from the sample 106. In some embodiments, the light source may transmit as the optical sensor is moved to the new position(s) (e.g., the light source continues to transmit light and does not deactivate and re-transmit light at different positions).

In some embodiments, there are multiple optical sensors that receive light serially (e.g., one at a time) or at the same time (e.g., simultaneously or near simultaneously) without moving the optical sensors.

In various embodiments, there are multiple light sources and multiple optical sensors configured to be able to detect absorbed light and scattering simultaneously or near simultaneously.

The particle detection platform 104 may include machine learning (ML) and/or artificial intelligence (AI) models to detect evidence of particles of interest in the spectrometer scans. The particle detection platform 104 may be or include any number of digital devices. The particle detection platform 104 may receive the set of intensity values, process the set of intensity values as described herein, generate a particle interest detection notification, and provide the particle of interest (e.g., a pathogen) detection notification. In some embodiments, the particle detection platform 104 provides the particle of interest detection notification to the computing device. The computing device may be or include any number of digital devices. The computing device may be a laptop and may be connected to the particle detection platform 104 via a physical cable, such as a Universal Serial Bus (USB) cable.

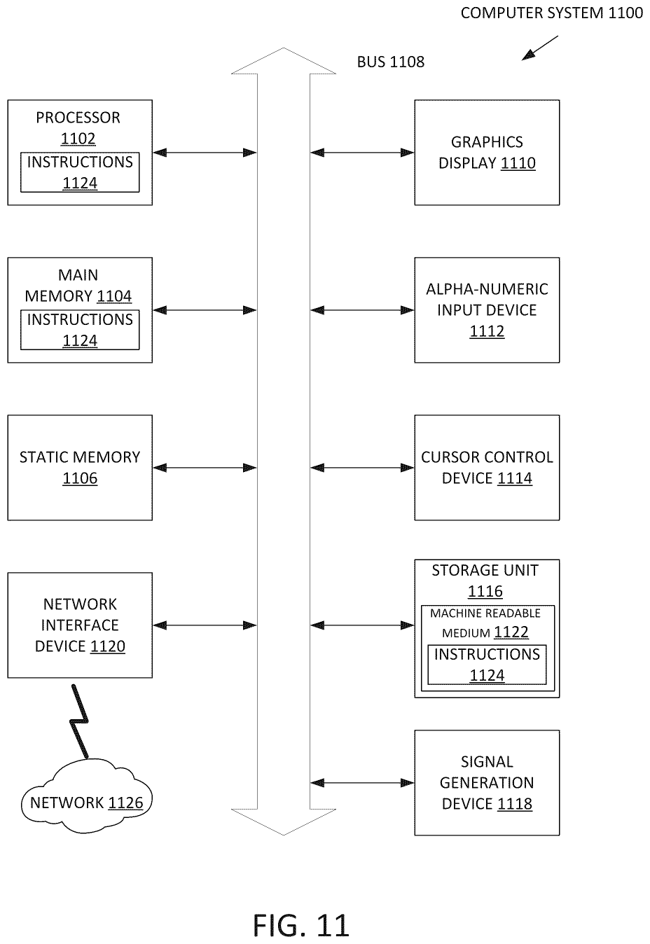

In some embodiments, the particle detection platform 104 and the computing device are not directly connected via a physical cable but are indirectly connected through a network, such as an IP-based Local Area Network (LAN), which may be part of or connected to the communication network 108. An example of a computing device may be seen in FIG. 11.

In some embodiments, the spectral acquisition system 102 and/or the particle detection platform 104 may provide notification to users tasked with safety, quality control, and/or regulators. In one example, the spectral acquisition system 102 and/or the particle detection platform 104 may, in the event of a positive foodborne pathogen detection notification, notify third-party systems such as those operated by food processors, those operated by government agencies such as the U.S. FDA, and/or those operated by third parties approved by such government agencies. In such an event, the spectral acquisition system 102 and/or the particle detection platform 104 may also recommend further diagnostic analysis by government agencies or other third parties approved by the government agencies.

In some embodiments, communication network 108 represents one or more computer networks (for example, LANs, WANs, and/or the like). The communication network 108 may provide communication between any of the spectral acquisition system 102 and particle detection platform 104. In some implementations, the communication network 108 comprises computer devices, routers, cables, and/or other network topologies. In some embodiments, the communication network 108 may be wired and/or wireless. In various embodiments, the communication network 108 may comprise the Internet, one or more networks that may be public, private, IP-based, non-IP based, and so forth.

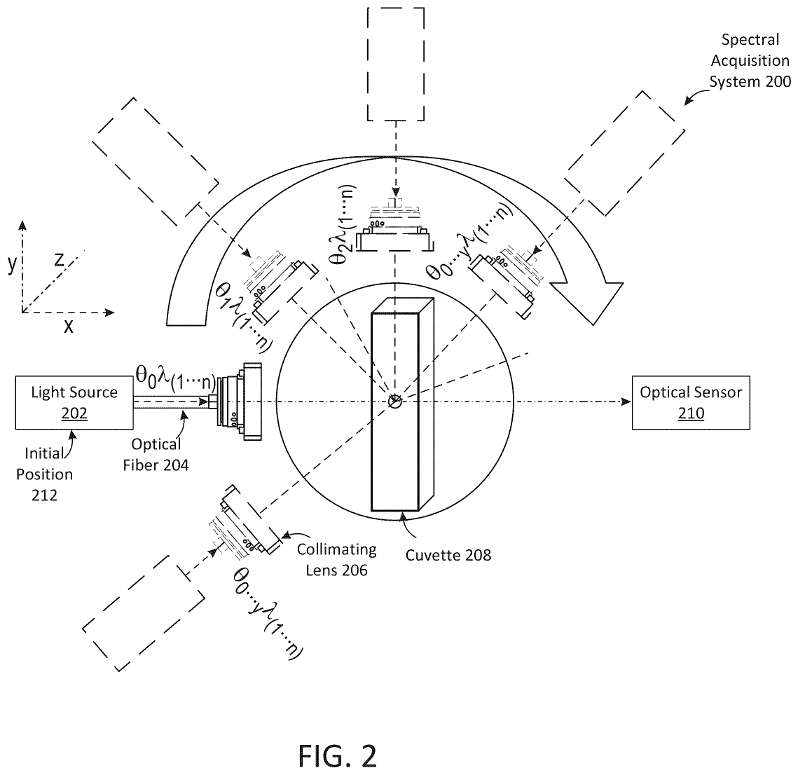

FIG. 2 depicts an example spectral acquisition system 200 with configurable angles of incidence detection according to some embodiments. The spectral acquisition system 200 includes a light source 202 at an initial position 212, an optical fiber 204, a collimating lens 206, a cuvette 208, and an optical sensor 210. The spectral acquisition system 200 may perform scans of samples for particles of interest (e.g., food processing byproducts) and obtain intensity measurements of a set of wavelengths of light that have passed through the samples.

In some embodiments, the light source 202 is an incoherent light source or a coherent light source. An incoherent light source outputs electromagnetic energy, or light, with a broad spectrum, where light waves have random phases and are not synchronized. In one example, the light source 202 may be, but is not limited to, a tungsten halogen bulb or one or more light-emitting diodes (LEDs). In one example, the light source 202 is a broadband light source. Broadband light sources produce light across a wide range of wavelengths. In contrast, coherent light sources produce light in a fixed wavelength or a narrow range of wavelengths.

The optical fiber 204 may couple the light source 202 to the collimating lens 206. In some embodiments, there is not an optical fiber 204 but rather the light source 202 transmits light through the collimating lens\206.

In various embodiments, the spectral acquisition system 200 includes a grating (not shown). The grating may separate spectral components of the light source. In one example, the grating may be a part of the collimating lens 206. In some embodiments, the grating may be optional.

In some embodiments, a light filtration apparatus may be positioned between the light source and the cuvette 208 or behind the cuvette 208 (e.g., relative to the transmittance of the light source). The light filtration apparatus may include an filter which absorbs unwanted wavelengths or interference filters which removes selected wavelengths by internal destructive interference and reflection.

The collimating lens 206 may expand, focus, and/or collimate the output of the light source 202 before being directed to the cuvette 208.

The contents of the cuvette 208 may include any type of sample. Some embodiments described herein discuss performing spectral analysis on water samples (e.g., obtained from wash water). In one example, the spectral acquisition system 102 and/or the particle detection platform 104 could perform spectral analysis on any food processing byproduct. Examples of food processing byproducts include, but are not limited to, water, wash water, oils, greases, animal blood, meat, and feces from animals such as cows, pigs, and chickens. Furthermore, samples may be obtained by swabbing or otherwise sampling food processing equipment, surfaces, residues, or anything that comes into contact with food. It will be appreciated that food processing byproducts are not limited to the examples described herein.

The optical sensor 210 may convert the intensity measurements in the set of intensity measurements to other values, such as absorbance values, transmittance values, or concentration values, and thus obtain a first set of values based on the set of intensity measurements. The optical sensor 210 may transmit the set of intensity values to the particle detection platform 104. In some embodiments, the spectral acquisition system 102 transmits the set of intensity measurements to a computing device.

In some embodiments, the spectral acquisition system 102 measures the detected light in units other than intensity, such as in absorbance units or transmittance units, and transmits the measured other values to the computing device. In some embodiments, the spectral acquisition system 102 transmits the first set of intensity values to the particle detection platform 104. In some embodiments, the spectral acquisition system 102 transmits the first set of intensity values to both the computing device and the particle detection platform 104.

The light source 202 may be an incoherent light source which provides light with multiple wavelengths and random phases. These light waves of varying wavelengths may be collimated by the collimating lens 206 and directed towards the sample 106 in the cuvette 208. In some embodiments, the pathogen sample 106 may be placed in a cuvette 208 and/or positioned in a receptacle of the cuvette 208. The optical sensor 210 may receive measurements of light absorbance, reflectance, and measurements of dynamic light scattering and convert the measurements into intensity measurements. The spectral acquisition system 200 may store the intensity measurements and the wavelength of light associated with the intensity measurement during the course of an initial scan. An angle of incidence of the light source 202 relative to the optical sensor 210 may be varied between an initial scan and subsequent scans scan of electromagnetic energy from the light source 202 by the optical sensor 210.

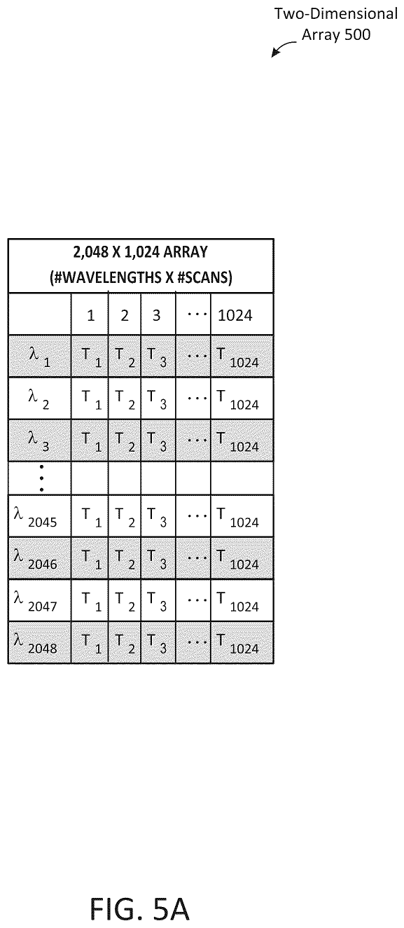

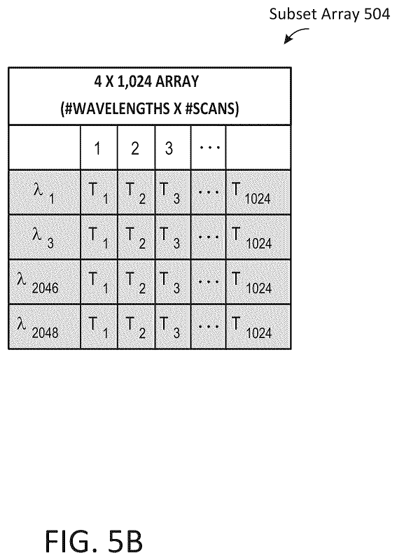

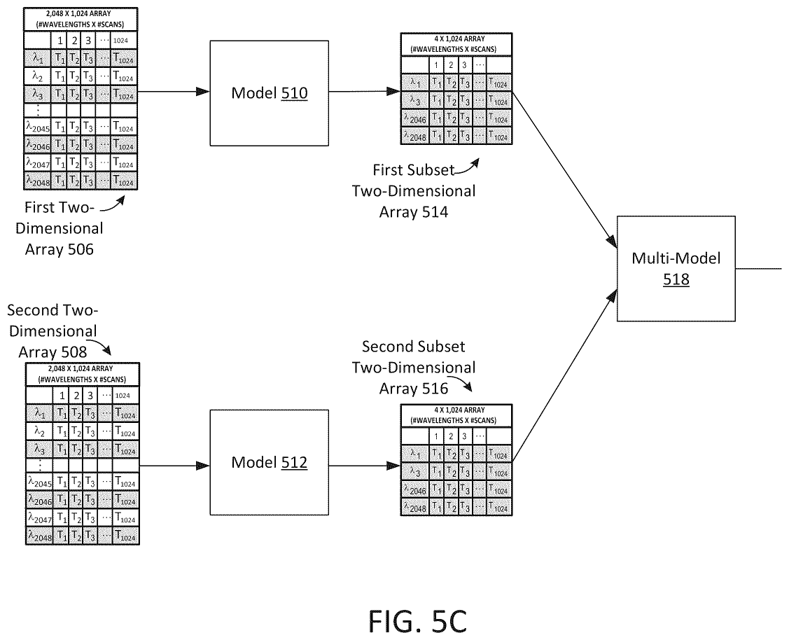

The result of the initial scan may be used to create an array (e.g., a two-dimensional array) of the wavelength of light detected by the optical sensor 210, an intensity measurement associated with the wavelength of light, and/or the number of times the wavelength of light was detected by the optical sensor 210 over the course of the initial scan. An example of the two-dimensional array 500 can be found in FIG. 5A.

In the illustrated embodiment of FIG. 5A, 2048 different wavelengths were detected over the course of an initial scan, and 1024 intensity measurements were recorded for each of the 2048 wavelengths. The two-dimensional array may be sent to the particle detection platform 104 for processing. It will be appreciated that there may be any number of wavelengths (e.g., more or less than 2048) detected over the course of any number of scans.

A two-dimensional array, such as the two-dimensional array 500 of FIG. 5A may be created for each configuration of the spectral acquisition system 200, which may results in an angle (or a variety of angles) of incidences between the light source 202 relative to the optical sensor 210. The reflection, absorption, and/or transmission of each wavelength of light may contain information that is pertinent to the identification or detection of a particular pathogen. However, if multiple configurations of the spectral acquisition system 200 are performed, the information captured by a single wavelength may vary over time.

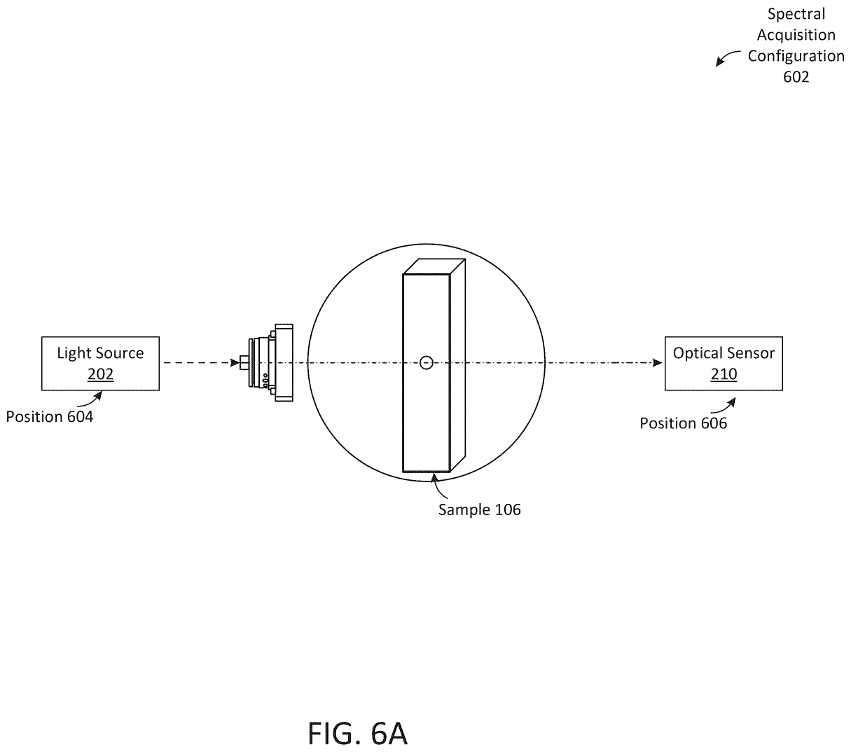

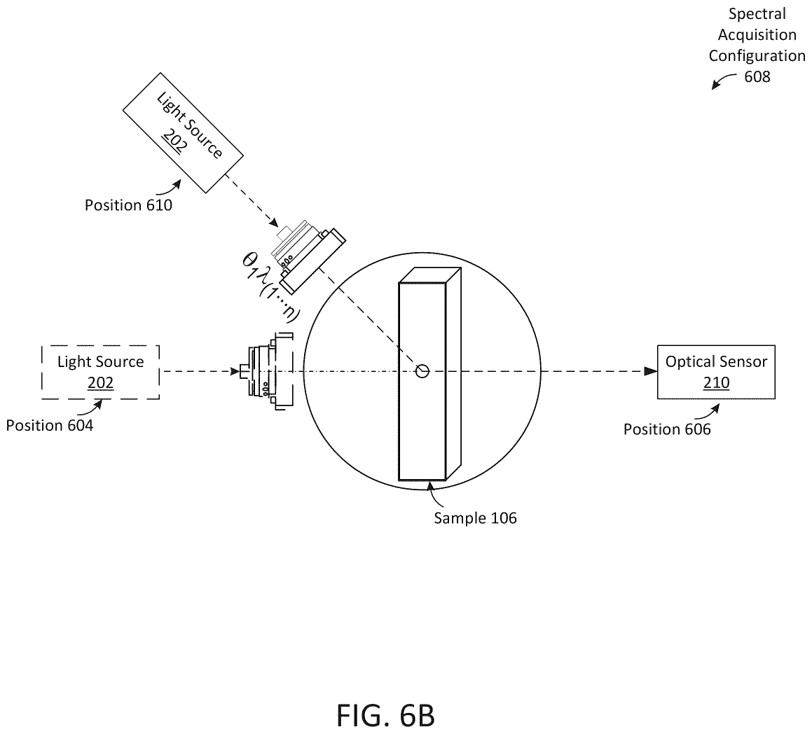

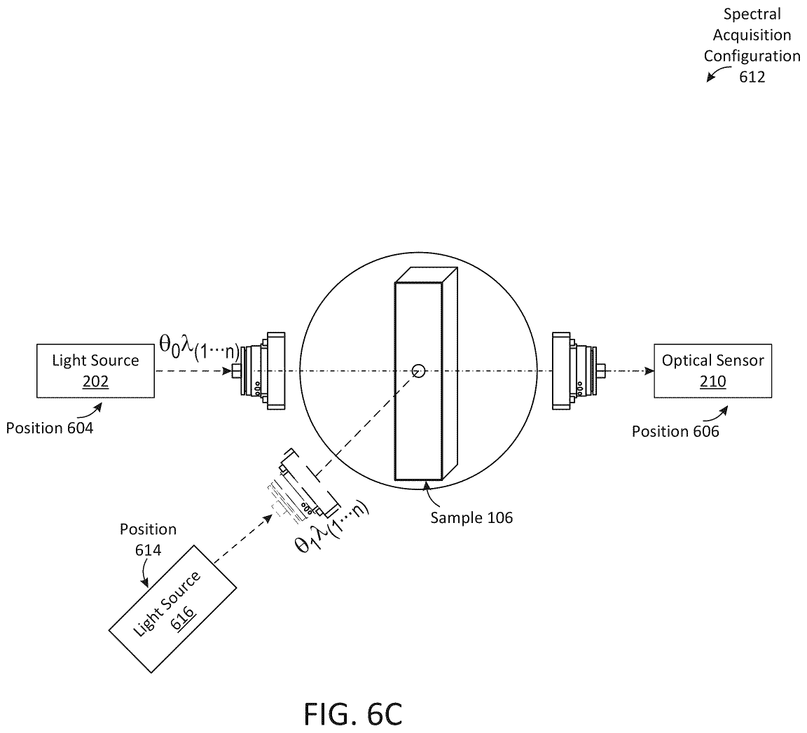

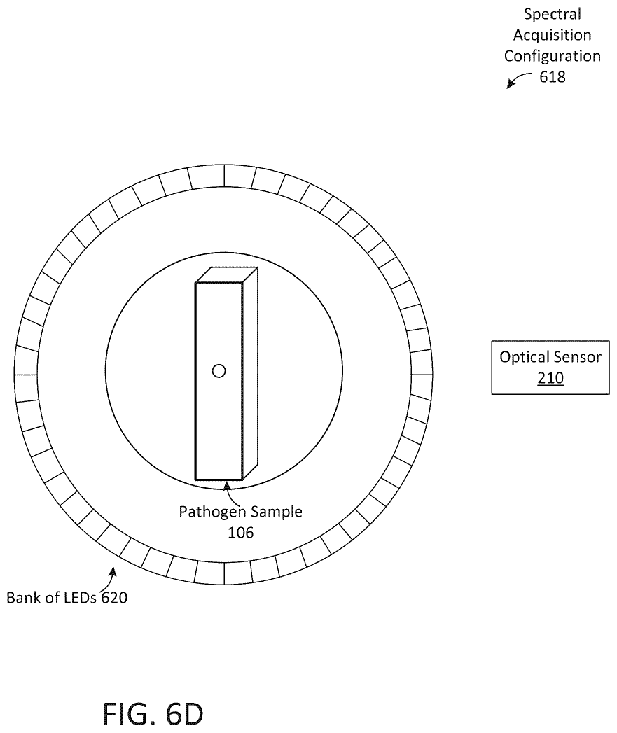

In some embodiments, the angle of incidence may be dynamically configurable in a number of ways. For example, the spectral acquisition system 102 may include a single light source 202. The single light source 202 may be repositioned (e.g., moved) between successive scans. An example of this configuration of the spectral acquisition system can be seen in FIG. 6B where a light source 202 is moved after transmission of light through the sample from position 604 to position 610 and the light source 202 again transmits light to the sample but the optical sensor 210 at position 606 makes measurements based on the angle of reflected light (e.g., scattering).

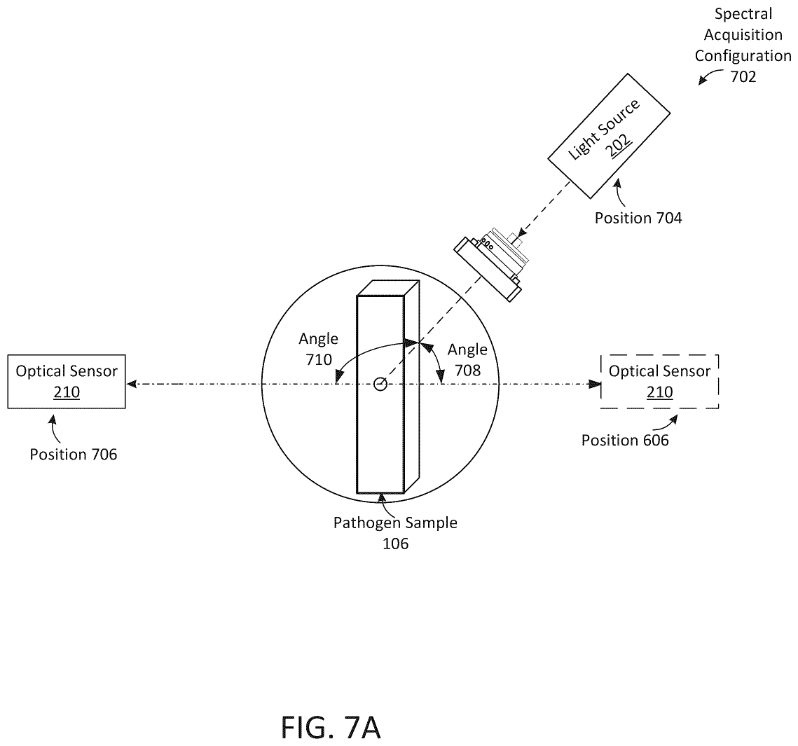

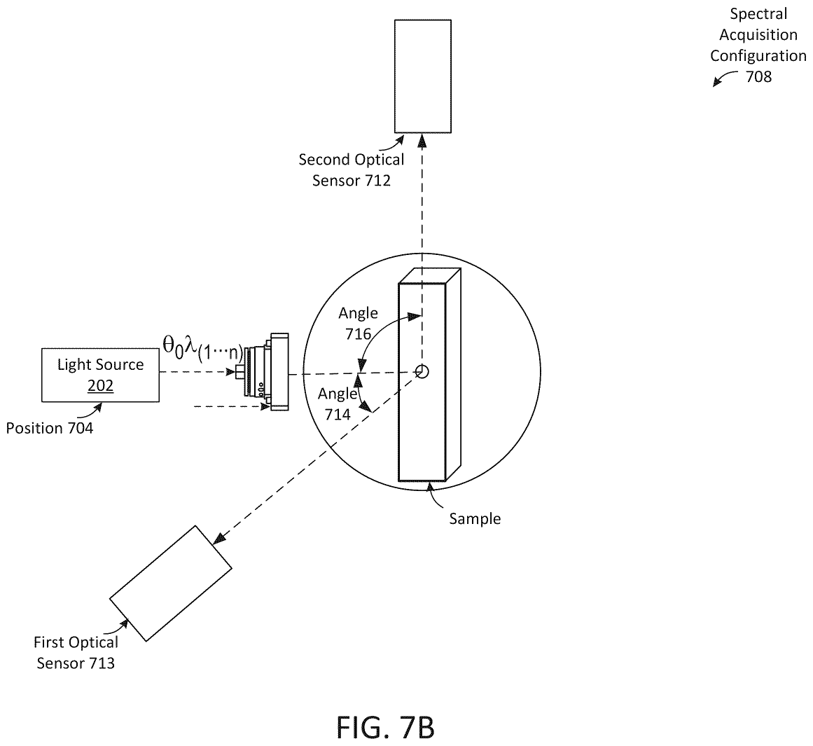

It will be appreciated that in other configurations, components (e.g., light source, the cuvette, or the optical sensor) may be repositioned (e.g., repositioned at least once to obtain three two-dimensional arrays, each with an angle of incidence that is different from the other). In another example, the spectral acquisition system 102 may include a single optical sensor 210. The single optical sensor 210 may be repositioned between successive scans. An example of this configuration of the spectral acquisition system 102 may be found in FIG. 7A. In some examples, multiple light sources 202 and/or multiple optical sensors 210 may be utilized.

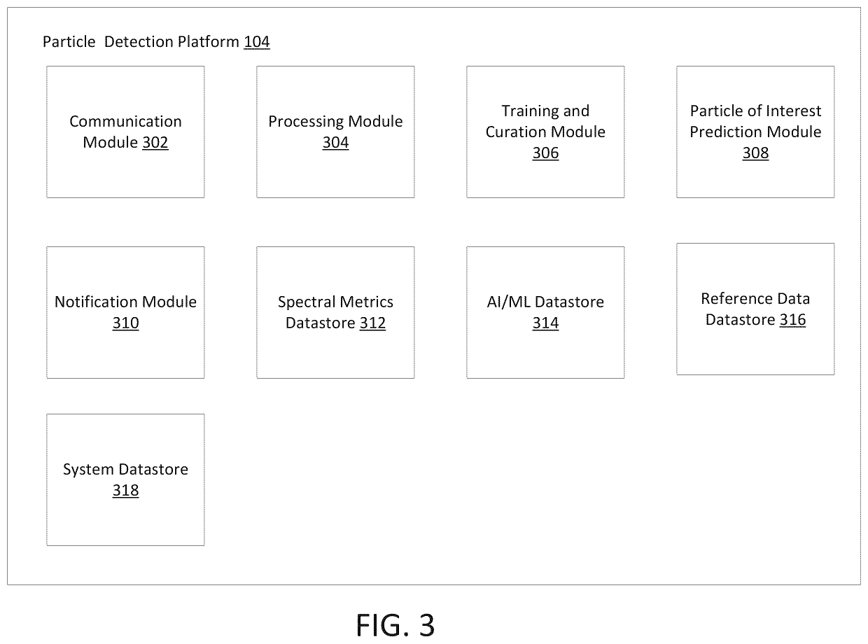

FIG. 3 depicts components of a block diagram of the particle detection platform 104 in some embodiments. The particle detection platform 104 includes a communication module 302, a processing module 304, a training and curation module 306, a particle of interest prediction module 308, a notification module 310, a spectral metrics datastore 312, an AI/ML datastore 314, a reference datastore 316, and a system datastore 318.

The communication module 302 may send and/or receive requests and/or data between the spectral acquisition system 102, the particle detection platform 104, and the computing device.

The processing module 304 may receive a request from the spectral acquisition system 102 for data, such as raw data from the spectral acquisition system 102. In one example, the raw data may include one or more two-dimensional arrays that may be transmitted to the processing module 304. The processing module 304 may receive the information (e.g., the two-dimensional array associated with a configuration of the spectral acquisition system 102) and input the information into a first previously trained model (e.g., previously trained by the training and curation module 306). In one example, the output of the first model may be a subset of the two-dimensional array. in this example, the subset of the two-dimensional array may include wavelengths in which intensity measurements over a particular threshold that were recorded for each wavelength in the subset of the two-dimensional array.

Another, separately trained model may receive information from different configurations of the spectral acquisition system 102. Each model may be trained based on or using a different configuration of the spectral acquisition system 102 such that specific models may be trained on the energy the sensor detects (e.g., scattering radiation based on light reflected from the sample at an angle as discussed herein). Each model may output information which is then provided to a meta-model which utilizes the output from the individual models to detect particles of interest.

The model may be any machine learning model, artificial intelligence model, and/or probability model. In some examples, each model may be a separately trained convolutional neural network or decision trees (e.g., gradient boosted decision trees).

The training and curation module 306 may train an artificial intelligence and/or machine learning system (e.g., such as a set of decision trees) to be applied to the measurements based on the transmission of light through or to the sample (e.g., the multiple two-dimensional arrays, each of the multiple two-dimensional arrays being associated with a configuration of the spectral acquisition system 102). The training AI/ML learning system may output measurements that indicate intensity, wavelengths of interest, size of particle, and/or presence of particle(s) of interest. In some embodiments, the wavelengths of interest represent wavelengths in which intensity measurements over a particular threshold.

The particle of interest prediction module 308 may apply the trained model(s) (e.g., such as the set of trained decision trees) to the different intensity values (e.g., measurements of wavelengths at different angles), each corresponding to a configuration of the spectral acquisition system 200.

The particle of interest prediction module 308 may include a multi-model (e.g., multi-layer model) that receives the output of two or more trained models and outputs measurements that may include or be used to generate a predicted particle class and predicted particle size. The predicted particle class may provide the name of the type, classification, or major group of a predicted pathogen. The output of the particle of interest prediction module 308 may include an estimated size or an estimated range of size of the predicted particle(s) of interest.

The particle of interest prediction module 308 may output a confidence score associated with the predicted particle(s) of interest based on many factors, including the detected wavelengths indicating the presence of the particle(s) of interest and/or the estimated size or the estimated range of sizes of the particle(s) of interest. For example, the multi-model may determine the presence of listeria based on wavelengths detected that match a particular range of a fingerprint for listeria. The prediction module 308 may further determine that the size of the particle(s) (e.g., using the same multi-model) detected are further within the detected size of the particle(s) of interest to confirm or deny the presence of the pathogen. For example, if the prediction module 308 determines that detected wavelengths match with listeria and if the prediction module 308 determines particles that match the expected size of listeria (e.g., if a certain class of listeria has a general size of 0.5 μm to 4 μm) then the prediction module 308 may provide notification or report the presence of listeria in the sample. Similarly, if the prediction module 308 determines a likelihood of a particular particle of interest but the wrong size is detected, then the particle detection platform 104 may generate a notification of the inconsistency and/or generate a report indicating that the particle(s) of interest are not or are likely not present. In some embodiments, the particle detection platform 104 may provide a confidence score that may be impacted based on a comparison of the expected size of the particle(s) of interest relative to the size(s) of particle(s) detected in the sample.

The models may have been previously trained by the training and curation module 306. In some embodiments, the interest prediction module 308 may have been previously trained to detect particles of interest. Particles of interest may be pathogens and/or chemicals that affect the environment or human health and safety. In one example, a result may indicate either a positive (a positive particular of interest detection) or a negative (a negative particular of interest detection) for a foodborne pathogen for the sample of the food processing byproduct. In some embodiments, the particle(s) of interest include chemicals and/or substances that are desirable for manufacturing, environmental protection, health, or the like.

In various embodiments, the artificial intelligence technology enables significantly reduced limits of detection (LOD), well beyond the LOD of the spectrophotometer (or other spectral acquisition device) alone. LOD is classically limited by the capabilities of the hardware and optical components. AI reaches beyond hardware and optical limitations.

The notification module 310 may generate and provide notifications that include results of particle(s) of interest detections of the sample and, optionally, other information, such as a confidence score. The notification module 310 may provide reports, alerts, and/or dashboards that include results, confidence scores, and/or other information. In one example, the notification module 310 provides the predicted name of the type, classification, or major group of detected pathogens.

In one example, the spectral acquisition system 102 may track pathogen detections on particular food processing equipment as well as what food was processed on the food processing equipment. As another example, the spectral acquisition system 102 may track foodborne pathogen detections in certain parts of a food processing facility as well as what food was processed in those certain parts. The particle detection platform 104 may thus be able to identify food (e.g., particular lots or production runs) and recommend, via the notification module 310, that remedial action, such as quarantining food, recalling food, or other action, should be taken. The notification module 310 may optionally notify appropriate third parties (e.g., government agencies such as the U.S. FDA) of the detection of foodborne pathogens. The notification module 310 may, in some embodiments, prepare reports to aid in compliance with food safety laws and regulations.

The spectral metrics datastore 312 may store raw data received from the spectral acquisition system 102, processed data (e.g., generated features), input for any number of models, output from any number of models, input to one or more multi-models, output from one of more multi-models, notifications, alerts, and/or the like. A data store is any data structure (e.g., one or more tables, databases, and/or the like) for storing information. In some embodiments, the information may be stored for auditing purposes. In some embodiments, the spectral metrics datastore 312 receives data from processing module 304. The data from the processing module 304 may include multiple subsets of the two-dimensional array, each of the two-dimensional arrays being associated with a configuration of the spectral acquisition system 102. The spectral metrics datastore 312 may include any number of data storage structures such as tables, databases, lists, and/or the like. An example of a subset of the two-dimensional array is an example subset array 504 of FIG. 5B. The rows of the subset array 504 include wavelengths of interest from the two-dimensional array 500 of FIG. 5A.

The AI/ML datastore 314 stores artificial intelligence/machine learning models (e.g., a set of decision trees) used to detect evidence of foodborne pathogens in the spectrometer scans. In some embodiments, the AI/ML datastore 314 stores the results of the artificial intelligence/machine learning models. In some embodiments, the artificial intelligence/machine learning models are stored on the particle detection system 104, which may be a cloud-based application. In one example, the artificial intelligence/machine learning models are stored on edge devices. An edge device is a device that provides an opening or entry point into an enterprise network. For security reasons, some corporations may require the artificial intelligence/machine learning models to be stored locally instead of storing the artificial intelligence/machine learning model in a cloud-based application that may be external to the corporation's enterprise network.

The reference datastore 316 stores reference data and metadata associated with the spectrometer data, spectrometer, and properties associated with the data.

In some embodiments, the reference datastore 316 includes configuration data associated with spectral acquisition system 200. Configuration data may include a number of different configurations of the spectral acquisition system 102 utilized in association with detecting pathogens from a sample. Configuration data may include the number and location of various components of the spectral acquisition system 102.

In a spectral acquisition configuration 612 of FIG. 6C, the spectral acquisition system may include light sources 202 and 616 and the optical sensor 210, as seen in FIG. 6C. The light source 202 may be located in position 604, while the light source 616 is located in a position 614. In some embodiments, the light source 202 may position an angle of incidence of 180 degrees or substantially 180 degrees to the optical sensor 210. The position 606 of the optical sensor 210 may be fixed or moveable. The position 614 may be different from that of the position 604. During an initial scan, light source 202 may be turned on while light source 616 remains off. The optical sensor 210 may receive a set of values corresponding to the measured intensity for the set of wavelengths of light from the light source 202. The angle of incidence may be able to allow for measurements of light scattering and/or reflectance (e.g., to create measurements of dynamic light scattering at the optical sensor 210 at position 606). During a subsequent scan, the light source 202 may remain off while the light source 616 is turned on. The optical sensor 210 may receive a set of values corresponding to the measured intensity for the set of wavelengths of light from the light source 616.

In one example, metadata may include a scan universal unique identifier, an external reference identifier, a specimen scanned data timestamp, which may be the date and time at which the spectral acquisition apparatus obtains the set of spectral metrics, spectral data file name, target particle or pathogen, inference request type name, operator ID, scan mode type code or name, medium name, modality name, device model, scan code or version code, device ID, location code name, customer ID, software version number code, and device firmware version identifier.

The scan universal unique identifier may be an identifier or identification number associated with a particular sample or specimen.

The external reference identifier may be a unique identifier for a particular sample assigned by a customer. The external reference identifier may link to a particular data file and metadata associated with the particular sample. The external reference identifier may enable a chain of custody, which may be used for tracking purposes.

The specimen scanned data timestamp may include a date and timestamp of when the sample or specimen was scanned by the computing device.

The spectral data file name may be the name of the spectral data file.

The target particle or pathogen may be a name of the pathogen or particle that the particle detection system 104 is trying to detect. In some embodiments, the target particle or pathogen may be E. Coli, Salmonella, or listeria, or an infectious pathogen of humans such as Respiratory Syncytial Virus (RSV), or Coronavirus (COVID-19).

In one example, the operator ID may be an internal ID number or name associated with a particular user of the spectral acquisition system 102 or laboratory technician.

Metadata associated with the scan mode type code or name may specify whether the sample or specimen was scanned in transmission mode or absorption mode. The results of scans in transmission mode may be expressed as a percentage or ratio (% T). The results of scans in absorption mode may be expressed in Absorption Unit (AU). In one example, for Hach spectrophotometers, to convert from Absorption Unit to transmission ratio, the following equation may be utilized:

% T=antilog(2−AU)

In various embodiments, the medium name identifies the medium in which the target particle is prepared for a scan. Examples of medium names may include DI Water, phosphate buffer solution (PBS), saline water, CITOSWAB®.

In another example, the modality name may refer to where the target particle was collected from. Examples of modality names include low nasal swabs, upper nasal swabs, oral swabs, oral rinse, urine, and blood sample.

The device mode may refer to whether the spectral metrics is processed by the artificial intelligence and/or machine learning system right away or if the spectral metrics is not processed right away.

In some embodiments, the customer ID is an identifier for each customer of the particle detection platform 104.

The software version number code and device firmware version identifier may represent or identify the version of the software being used.

The system datastore 318 stores the results of foodborne pathogen detections of the sample as well as other information, such as a confidence score. Reports and/or dashboards that include results, confidence scores, and/or other information may be stored in the system datastore 318.

A module of the computing device or the particle detection platform 104 may be hardware, software, firmware, or any combination. For example, each module may include functions performed by dedicated hardware (e.g., an Application-Specific Integrated Circuit (ASIC), a Field Programmable Gate Array (FPGA), or the like), software, instructions maintained in ROM, and/or any combination. Software may be executed by one or more processors. However, a limited number of modules are depicted in FIG. 3, there may be any number of modules. Further, individual modules may perform any number of functions, including functions of multiple modules, as shown herein. Further, modules depicted as being included in the computing device may be additionally or alternatively included in the particle detection platform 104, and modules included in the particle detection platform 104 may be additionally or alternatively included in the computing device.

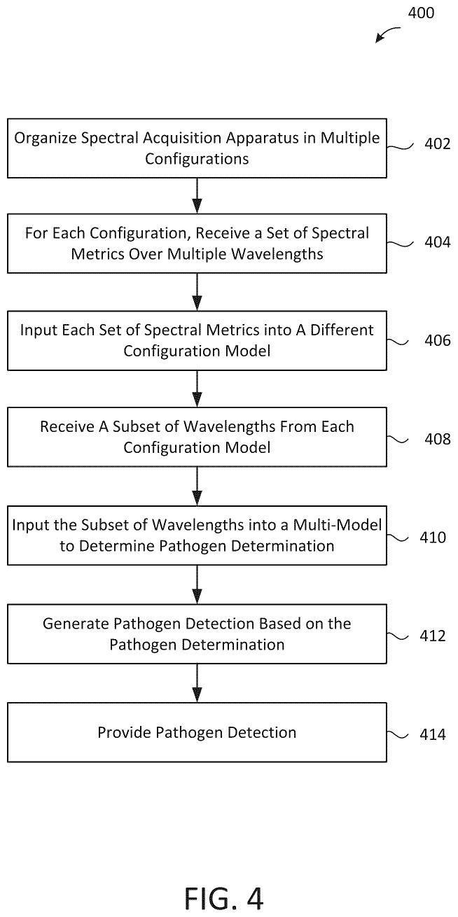

FIG. 4 is a flowchart showing a method 400 for detecting particles of interest in some embodiments. Various modules of the particle detection platform 104 perform the method 400. In some embodiments, various modules of computing device performs the method 400. In some embodiments, various modules of both the computing device and the particle detection platform 104 perform the method 400.

The following example is directed to detecting food-based pathogens, although it will be appreciated that the method may be utilized to detect particles of interest in many different contexts. In some embodiments, a person working in a food processing facility in which the pathogen sample 106 is located fills the cuvette 208 or other suitable container with a sample of food processing byproduct and places the cuvette 208 in an appropriate receptacle of the spectral acquisition system 102. The person may fill the cuvette 208 periodically, as needed, for each lot or shipment of food to be processed or on a predetermined schedule. It will be understood that samples of food processing byproducts may be tested at various times. In some embodiments, the cuvette 208 may be filled by an automated device or system without intervention by a person. In some embodiments, the sample of food processing byproduct may be mixed with a reagent. In some embodiments, the sample of food processing byproduct may be mixed with a neutral or inert substance.

After filling the cuvette 208, the person may then start a scan of the sample of the food processing byproduct using an interface of the spectral acquisition system 102. Additionally, or alternatively, the person may start the scan using the computing device, which may control the spectral acquisition system 102. The spectral acquisition system 102 generates light that passes through at least a portion of the sample of the food processing byproduct in the cuvette 208 and detects the light that has passed through at least the portion of the sample of the food processing byproduct in the cuvette 208. The spectral acquisition system 102 measures the intensities of the detected light for a set of wavelengths of the light and obtains a set of intensity measurements for the set of wavelengths of the light.

The method 400 may begin at step 402, and the spectral acquisition system 102 may be configured in a number of ways. In some embodiments, instead of having multiple light sources, such as the spectral acquisition configuration 612 of FIG. 6C or repositioning the light source in subsequent scans, such as a spectral acquisition configuration 608 of FIG. 6B. The spectral acquisition system 102 may include multiple light emitting diodes (LEDs) such as a bank of LEDs 620 as seen in a spectral acquisition configuration 618 of FIG. 6D. The LEDs 620 may include a light-emitting diode (LED) arranged in a circular structure. Different LEDs of the LEDs 620 may be turned on to produce different angles of incidence between the light source (one LED of the LEDs 620) and the optical sensor 210. Different LEDs of the LEDs 620 may be turned on between successive scans of the pathogen sample 106.

In one example, the spectral acquisition system 102 may have a configuration similar to that of a spectral acquisition configuration 702 of FIG. 7A. The optical sensor 210 may be located in the position 606, the light source 202 may be located in a position 704 during an initial scan. An angle 708 represents an angle of separation between the light source 202 and the position 606 of the optical sensor 210. During a subsequent scan, the optical sensor 210 may be located in a position 706. An angle 710 represents an angle of separation between the light source 202 and the position 706 of the optical sensor 210.

In step 404, the communication module 302 receives a set of intensity values from the spectral acquisition system 102. In some embodiments, the communication module 302 receives a different set of intensity values associated with each configuration of the spectral acquisition system 102. In various embodiments, the set of intensity values may be based on the set of intensity measurements for a set of wavelengths of light that the spectral acquisition system 102 obtained. For example, the spectral acquisition system 102 may be configured in a way similar to that of a spectral acquisition configuration 602 of FIG. 6A. In spectral acquisition configuration 602, the light source 202 and the optical sensor 210 may be positioned in such a way that the angle of incidence between the two components of the spectral acquisition system 102 is 180 degrees or substantially 180 degrees. With each successive spectral acquisition configuration, where the angle of incidence between the light source 202 and the optical sensor 210 can be achieved in any number of ways, some of which have been previously discussed in FIG. 6A through 6D. In this series of figures, the number of light sources and/or the position of light sources are changed between successive scans.

The communication module 302 may receive a first two-dimensional array 506 of FIG. 5C from the spectral acquisition system 102. The first two-dimensional array 506 may be associated with an initial scan of the spectra acquisition configuration 702 of FIG. 7A, in which the optical sensor 210 is located in position 606, and the light source 202 is located in the 706. This may represent an initial scan of the light source 202 of the pathogen sample 106.

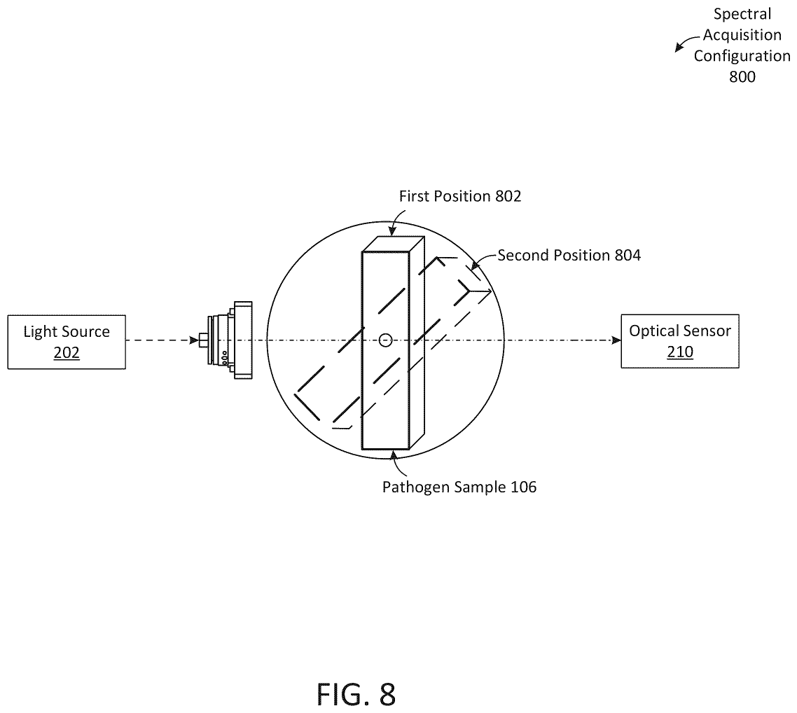

In the example of FIG. 7A, the position 704 of the light source 202 is fixed, while the position of the optical sensor 210 is changed or varied with successive scans. In the illustrated embodiment, two scans of the pathogen sample 106 are depicted. It can be appreciated that the optical sensor 210 may be located in any number of positions to vary the angle of incidence between the light source 202 and the optical sensor 210. In one example, the number and location of the light source 202 and the optical sensor 210 may remain constant. In such an example, the position of the pathogen sample 106 may be changed or repositioned. In a spectral acquisition configuration 800 of FIG. 8, the pathogen sample 106 may repositioned from a first position 802 during an initial scan to a second position 804 in a subsequent scan of the pathogen sample 106 by the light source 202. Although FIG. 8 depicts the pathogen sample 106 rotating to different positions, the system may move the pathogen sample 106 in any direction (e.g., forward, backward, laterally, upwards, downwards, at an angle, and/or the like). In some embodiments, the system may shake the pathogen sample 106 to cause movement or change in the sample. In some embodiments, the system may stabilize the sample to dampen vibrations and/or let the sample settle before taking measurements.

FIG. 7B depicts a configuration of a spectral acquisition system where multiple sensors are utilized to acquire spectral measurements according to some embodiments. In this example, the position 704 of the light source 202 is fixed, while there are two optical sensors 712 and 713. The position of the optical sensors may also be fixed. There may be any number of optical sensors including, for example, an optical sensor located directly across from the light source 202 relative to the sample. In this example, the angle 716 represents an incident angle between the transmission of light from the light source 202 to the sample relative to the detected energy by the second optical sensor 712. Similarly, the angle 714 represents an incident angle between the transmission of light from the light source 202 to the sample relative to the detected energy by the optical sensor 713.

In this example, the light source may transmit light to the sample and the first and second optical sensors may detect light simultaneously.

Measurements by the first optical sensor 710 may be further processed and analyzed by a trained model (e.g., decision trees or CNN) that is trained on receiving data at this or similar angles (e.g., similar to angle 716). Similarly, measurements by the second optical sensor 712 may be further processed and analyzed by a different trained model (e.g., decision trees or CNN) that is trained on receiving data at this or similar angles (e.g., similar to angle 714). The output of the models may or may not be further processed before being received by the meta-model. The meta-model may be trained based using training data from the trained first and second model to detect particle(s) of interest and/or particle size. The output of the meta-model may be used to classify detection of particle(s) of interest based on detected wavelength intensities (e.g., if they match expected wavelength intensity(ies) of particle(s) of interest and/or are the correct size relative to expected particle size for the particle(s) of interest.

At step 406, the processing module 304 applies a set of trained decision trees to the set of intensity values received in step 404. A separate set of intensity values may be associated with each configuration of the spectral acquisition system 102. The first two-dimensional array 506 associated with the spectral acquisition configuration 702 may be inputted to a model 510 of FIG. 5C. The first two-dimensional array 506 is associated with an initial scan of the spectral acquisition configuration 702. The second two-dimensional array 508, associated with a subsequent scan of the spectral acquisition configuration 702 may be inputted to a model 512 of FIG. 5C.

In step 408, the AI/ML system of the processing module 304 may output indicating detected wavelengths, features, or other information determined by the model. In some embodiments, the output from the model may include wavelengths in which intensity measurements over a particular threshold were recorded for each wavelength in the subset of the two-dimensional array. The AI/ML system may be previously trained by the training and curation module 306.

The output of the model 510 may be a first results while the output of the model 512 may be second results 514 516.

In step 410, the communication module 302 may send a request to the particle of interest prediction module 308 to receive the results 514 and 516 and apply them to a multi-model for further analysis (e.g., the multi-model 518 of FIG. 5C).

In step 412, the particle of interest prediction module 308 may include a multi-layer model that receives multiple subsets of the two-dimensional array and outputs a predicted particle class and predicted particle size. The predicted particle class may provide the name of the type, classification, or major group of predicted pathogen. The output of the particle of interest prediction module 308 may include an estimated size or an estimated range of size of the predicted pathogen. The particle of interest prediction module 308 may output a confidence score associated with the predicted pathogen based on many factors, including the estimated size or the estimated range of size of the predicted pathogen. For example, if a certain class of listeria has a general size of 0.5 μm to 4 μm, then having models associated with listeria predict a similar size along with the class prediction of listeria will output a greater confidence score.

At step 412, the particle of interest prediction module 308, based on the result, determines either a positive foodborne pathogen detection or a negative foodborne pathogen detection for the foodborne pathogen in the sample of the food processing byproduct. In some embodiments, the particle of interest prediction module 308 determines that the result indicates a positive foodborne pathogen detection if the result meets or exceeds a threshold and that the result indicates a negative foodborne pathogen detection if the result does not meet or exceed the threshold. In embodiments where the result is a float value between zero, inclusive, and one, inclusive, the threshold may be 0.5. In embodiments where the result is an integer with a value of either zero, one, or another integer value greater than one, zero indicates a negative result and one or another integer value greater than one indicates a positive result. As discussed in more detail herein, in such embodiments, the result may indicate both a positive foodborne pathogen detection as well as a concentration of the foodborne pathogen in the sample of the food processing byproduct.

At step 414, the notification module 310, based on the result, determines an approximate concentration or an approximate range of concentrations for the foodborne pathogen in the sample of the food processing byproduct. In some embodiments, the notification module 310 provides a confidence value for the foodborne pathogen in the sample of the food processing byproduct.

The notification module 310 may generate a foodborne pathogen detection notification that indicates either the positive foodborne pathogen detection or the negative foodborne pathogen detection for the foodborne pathogen in the sample of the food processing byproduct. In some embodiments, the foodborne pathogen detection notification further indicates the approximate concentration or the approximate range of concentrations for the foodborne pathogen in the sample of the food processing byproduct. In some embodiments, the foodborne pathogen detection notification further indicates the confidence value for the foodborne pathogen in the sample of the food processing byproduct. In some embodiments, if the confidence value is within a certain range or above or below a certain threshold, the foodborne pathogen detection notification may include a flag indicating such. For example, if the confidence value is below a certain threshold, the foodborne pathogen detection notification may flag that there is low confidence in the result. As another example, if the confidence value is above a certain threshold, the foodborne pathogen detection notification may flag that there is high confidence in the result.

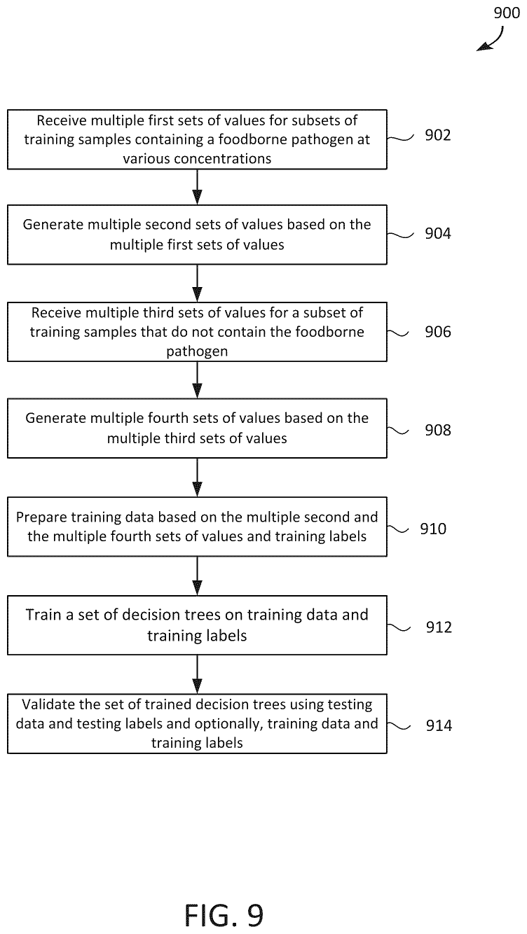

FIG. 9 is a flowchart showing method 900 for training sets of decision trees for detecting pathogens in some embodiments. Various modules of the particle detection platform 104 perform the method 900. In some embodiments, various modules of the computing device perform the method 900. In some embodiments, various modules of both the computing device and the particle detection platform 104 perform the method 900.

In some embodiments, a person, such as a laboratory technician, prepares a set of training samples. A foodborne pathogen, such as E. coli, may be cultivated in a solution, such as tryptic soy broth (TSB) solution. The initial concentration of the foodborne pathogen in the solution may be approximately 1e⁸ colony-forming units/milliliter (cfu/mL). In some embodiments, the initial concentration of the foodborne pathogen in the solution may range from approximately 1e⁶ to approximately 1e⁸ cfu/mL. In some embodiments, the initial concentration of the foodborne pathogen in the solution may be greater than approximately 1e⁸ cfu/mL. In some embodiments, the initial concentration of the foodborne pathogen in the solution may be lower than approximately 1e⁸ cfu/mL.

A first subset of training samples at the initial concentration of approximately 1e⁸ cfu/mL may be prepared. A second subset of training samples may be prepared that have been diluted 10:1 from the initial concentration using the solution, so as to have a second concentration of approximately 1e⁷ cfu/mL. A third subset of training samples may be prepared that have been diluted 100:1 from the initial concentration using the solution, so as to have a third concentration of approximately 1e⁶ cfu/mL. A fourth subset of training samples may be prepared that have been diluted 1000:1 from the initial concentration using the solution, so as to have a fourth concentration of approximately 1e⁵ cfu/mL. A fifth subset of training samples may be prepared that have been diluted 10,000:1 from the initial concentration using the solution, so as to have a fifth concentration of approximately 1e⁴ cfu/mL. A sixth subset of training samples may be prepared that have been diluted 100,000:1 from the initial concentration using the solution, so as to have a sixth concentration of approximately 1e³ cfu/mL. A seventh subset of training samples may be prepared that have been diluted 1,000,000:1 from the initial concentration using the solution, so as to have a seventh concentration of approximately 1e² cfu/mL. An eighth subset of training samples may be prepared that have been diluted 10,000,000:1 from the initial concentration using the solution, so as to have an eighth concentration of approximately 1 cfu/mL. A ninth subset of training samples may be prepared that contain only the solution, for example, the TSB solution.

In some embodiments, there may be fewer than or more than eight subsets of training samples at different concentrations. In some embodiments, the different subsets of training samples may be diluted using different dilution ratios to obtain different concentrations than those described herein. In some embodiments, the set of training samples contains approximately 2000 training samples that include a foodborne pathogen, which may be referred to herein as positive training samples. In some embodiments, the set of training samples contains fewer than 2000 positive training samples. In some embodiments, the set of training samples contains more than 2000 positive training samples. In some embodiments, the set of training samples contains approximately the same number of training samples that do not include a foodborne pathogen, which may be referred to herein as negative training samples, as the number of positive training samples. In some embodiments, the number of negative training samples is less than the number of positive training samples. In some embodiments, the number of negative training samples is more than the number of positive training samples.

In some embodiments, the set of training samples are prepared prior to the spectral acquisition system 102 scanning the training samples. In some embodiments, the first subset of training samples at the initial concentration of approximately 1e⁸ cfu/mL are prepared and scanned by the spectral acquisition system 102. Then, the first subset of training samples are diluted 10:1 from the initial concentration to obtain the second subset of training samples at the second concentration of approximately 1e⁷ cfu/mL, and then the second subset of training samples are scanned by the spectral acquisition system 102. This dilution and scanning may be repeated several times to obtain, and then scan, the third through eighth subsets of training samples.

To scan a training sample, the cuvette 208 or other suitable container may be filled with a training sample and placed in an appropriate receptacle of the spectral acquisition system 102. The person may then start a scan of the sample of the food processing byproduct using an interface of the spectral acquisition system 102. Additionally or alternatively, the person may start the scan using a computing device, which may control the spectral acquisition system 102. The spectral acquisition system 102 generates light that passes through at least a portion of the training sample in the cuvette 208 and detects the light that has passed through at least the portion of the training sample in the cuvette 208. The spectral acquisition system 102 measures intensities of the light for a set of wavelengths of the light and obtains a set of intensity measurements for the set of wavelengths of the light.

The method 900 begins at step 902 where the communication module 302 receives multiple first sets of intensity values from the spectral acquisition system 102 via the computing device based on multiple scans of the first subset of training samples containing the foodborne pathogen at the first concentration. The multiple first set of intensity values are based on multiple sets of intensity measurements for a set of wavelengths of light that the spectral acquisition system 102 obtained for scans of the first subset of training samples containing the foodborne pathogen at the first concentration.

It will be appreciated that method 900 may be used to train each different model based on angle of light detected by the detector. In one example, a first model may be trained on intensity values that are generated based on light detected by an optical sensor that is directly across from the sample and light source (e.g., an angle of 180 degrees that is measured along the light path from the light source, through the sample, to the detector). The second model may be trained on intensity values that are generated based on light detected by an optical sensor that is at an angle relative to light transmitted to the source. For example, light may be transmitted to a source and be reflected at an angle that is detected by the optical sensor (e.g., to detected scattering). Similarly, the meta-model may be trained using the output of the trained models using training data to detect any number of particles of interest.

Step 902 may be performed for each subset of training samples containing the foodborne pathogen at a different concentration. That is, the communication module 302 may perform step 902 for the first subset of training samples at the first concentration of approximately 1e⁸ cfu/mL, for the second subset of training samples at the second concentration of approximately 1e⁷ cfu/mL, up to and including for the eighth subset of training samples at the eighth concentration of approximately 1 cfu/mL.

Returning to FIG. 9, at step 904, the processing module 304 generates multiple second sets of intensity values based on the multiple first sets of intensity values. In some embodiments, the processing module 304 normalizes each value in the multiple second sets of values to be between zero, inclusive, and one, inclusive. The processing module 304 may further process the values in the multiple second sets of intensity values. The processing module 304 may generate the multiple second sets of values using other techniques, such as applying a fitting function to each value in the multiple first sets of values to generate each value in the multiple second sets of intensity values.

Step 904 may be performed for each subset of training samples containing the foodborne pathogen at a different concentration. That is, the processing module 304 may perform step 904 for the first subset of training samples at the first concentration of approximately 1e⁸ cfu/mL, for the second subset of training samples at the second concentration of approximately 1e⁷ cfu/mL, up to and including for the eighth subset of training samples at the eighth concentration of approximately 1 cfu/mL.

At step 906, the communication module 302 receives multiple third sets of intensity values from the spectral acquisition system 102 based on multiple scans of the subset of training samples that do not contain the foodborne pathogen (e.g., at different angles as discussed herein). At step 908 the processing module 304 generates multiple fourth sets of intensity values based on the multiple third sets of intensity values. In some embodiments, the processing module 304 normalizes each intensity value in the multiple fourth sets of intensity values to be between zero, inclusive, and one, inclusive. The processing module 304 may further process the intensity values in the multiple fourth sets of intensity values. The processing module 304 may generate the multiple fourth sets of intensity values using other techniques, such as applying a fitting function to each value in the multiple third sets of intensity values to generate each value in the multiple fourth sets of intensity values.

At step 910, the training and curation module 306 prepares training data based on the multiple second sets of intensity values and the multiple fourth sets of intensity values. The training and curation module 306 may also prepares training labels for the training data. In embodiments where the set of trained decision trees operate in a binary mode, a training label may be either a zero (0) for a negative training sample and a one (1) for a positive training sample. In embodiments where the set of trained decision trees operate in a multiclass mode, a training label may be either a zero (0) for a negative training sample, a one (1) for a positive training sample having a foodborne pathogen concentration at a first concentration, a two (2) for a positive training sample having a foodborne pathogen concentration at a second concentration, a three (3) for a positive training sample having a foodborne pathogen concentration at a third concentration, a four (4) for a positive training sample having a foodborne pathogen concentration at a fourth concentration, a five (5) for a positive training sample having a foodborne pathogen concentration at a fifth concentration, a six (6) for a positive training sample having a foodborne pathogen concentration at a sixth concentration, a seven (7) for a positive training sample having a foodborne pathogen concentration at a seventh concentration, and an eight (8) for a positive training sample having a foodborne pathogen concentration at an eighth concentration. In some embodiments, there are fewer than eight different concentrations of the foodborne pathogen in the training samples and a corresponding lower number of different training labels. In some embodiments, there are more than eight different concentrations of the foodborne pathogen in the training samples and a corresponding higher number of different training labels.

At step 912, the training and curation module 306 trains a set of decision trees (e.g., one or more models) for the foodborne pathogen. In some embodiments, the training and curation module 306 utilizes an optimized distributed gradient boosting library, XGBoost. In some embodiments, the training and curation module 306 utilizes the following Python code to create each set of decision trees:

```
  from xgboost import XGBClassifier
  params = {“booster”: “gbtree”,
    “objective”:“binary:logistic”,
    “max_delta_step”:20,
    “eval_metric”:“error”,
    “n_estimators”:10000,
    “verbosity”:0,
    “max_depth”:500,}
    self.config.params = params
model = XGBClassifier(**params)
```

XGBClassifier may be understood as a single model that is an ensemble of 10,000 decision trees (the “n_estimators”: 10000 parameter). In some embodiments, the training and curation module 306 may utilize parameters other than or in addition to those listed herein. In some embodiments, the training and curation module 306 may utilize different values for model parameters than those listed herein.

In some embodiments, the training and curation module 306 utilizes the following Python code to train each set of the multiple sets of decision trees:
model.fit(x_train, y_train, eval_set=[(x_train, y_train), (x_test, y_test)], early_stopping_rounds=50)

In this code, x_train is training data, y_train is training labels, x_test is testing data, and y_test is testing labels. Both x_train and x_test are ground truth data. Both x_train and x_test may include both positive training samples and negative training samples. In some embodiments, both the x_train and x_test data are balanced, meaning that they include equal or generally equal numbers of positive training samples and negative training samples. In some embodiments, the x_train and x_test data may be imbalanced toward negative training samples, meaning that they include more negative training samples than positive training samples. The training and curation module 306 may also use data sets that are imbalanced towards positive training samples, meaning that they include more positive training samples than negative training samples.

The set of trained decision trees may operate in a binary mode or a multiclass mode. The following Python code may be utilized to determine which mode the set of trained decision trees may operate in:

```
# if binary, set binary, otherwise, set multiclass
if np.max(y_train) == 1:
  params[‘objective’] = “binary:logistic”
  params [‘eval_metric’] = ‘error’
elif np.max(y_train) > 1:
  params [‘objective’] = “multi:softmax”
  params [‘eval_metric’] = ‘merror’
```

At step 914, the training and curation module 306 validates the set of trained decision trees. In some embodiments, the training and curation module 306 utilizes both training data and testing data to validate the set of trained decision trees. In some embodiments, the training and curation module 306 utilizes only testing data to validate the set of trained decision trees.

It will be appreciated that method 900 may be used to train models to detect many different kinds of particles and is not limited or required to be directed to food based pathogens. For example, method 900 may be directed to detecting desirable or undesirable chemicals, nutrients, substances, toxins, elements in the air, blood chemistry, viruses (e.g., COVID), and/or the like.

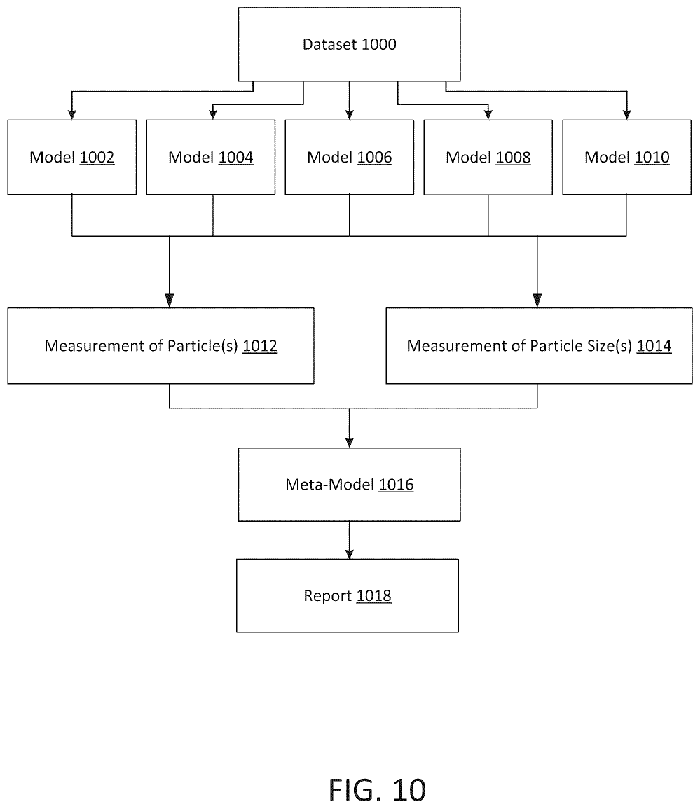

FIG. 10 depicts a high-level diagram of the multi-dimensional array and high-level models according to some embodiments. Dataset 1000 may include intensity measurements taken by an optical sensor at different angles relative to the light path. Each model may receive a different set of intensity values based on measurement of detected energy. Each model may be previously trained and configured to receive detected energy at a particular angle or range of angles discussed herein (e.g., the angle formed by transmission of light to the source and then to the optical sensor). For example, model 1002 may receive intensity measurements of light transmitted from the light source through the sample to the optical sensor (e.g., an angle of 180). Model 1002 may be used to detect absorption and/or transmission, for example. Model 1004 may receive intensity measurements of light transmitted from the light source to the sample and reflected (e.g., scattered) at a particular angle or range of angles. Model 1006 may receive intensity measurements of light transmitted from the light source to the sample and reflected (e.g., scattered) at a different angle or different range of angles that is different from those received by other models. Similarly for the other models 1008 and 1010, the models may each receive different intensity measurements reflected at further different angles (from each other and other models). There may be any number of models configured to receive different measurements by any number of optical sensor(s) (e.g., an optical sensor may move to obtain the different angle(s), there may be multiple optical sensor, and/or the optical sensor(s) may be stationary and other components of the system may create the angles as discussed herein).

Each model may be an AI model, probability model, analytics model, or a combination of the like. In one example, each model is a different set of trained decision trees.

The output of each model 1002-1010 may be a different measurement or prediction of detected substances or particles (e.g., particles of interest) 1012 based on the model and the particular intensity values received. Further, optionally or in some embodiments, the output of each model 1002-1010 may be a measurement or prediction of substance and/or particle size 1014 (e.g., particles of interest) based on the model and the particular intensity values received.

The meta-model 1016 may be trained on training data sent through the models 1002-1010 (or any number of models). The meta-model 1016 may be an AI model, probability model, analytics model, or a combination of the like. In one example, the meta-model 1016 is a different set of trained decision trees.

The meta-model 1016 is configured to receive the output from the models 1002-1010. In some embodiments, the output of any number of the models 1002-1010 may be further processed and/or normalized.

The meta-model 1016 may generate a report 1018 that includes measurements, an indication of likely particle(s) of interest detection, an indication of size of particle(s) detected, confidence score(s), and/or the like. In some embodiments, the output of the meta-model 1016 indicates whether particle(s) of interest are detected (e.g., based on the input from the other models 1002-1010 including wavelength measurements and/or particle sizes). The output may further include a degree of confidence. Alternately, the output of the meta-model 1016 may include wavelength intensities that are compared to expected wavelengths and/or expected particle sizes of particle(s) of interest to determine the presence or confidence of detection. The output of the meta-model 1016 may be optionally further processed, scaled, and/or normalized before further analysis and/or reporting (e.g., in the report 1018).

In some embodiments, the report 1018 may include or be in addition to an alert indicating the detection or lack of detection of particle(s) of interest. The alert may be provided to a digital device and/or user (e.g., via email, text, call or the like). In one example, the alert may indicate the presence of a pathogen (e.g., a person is infected with covid, the food byproduct has listeria, or there are toxins in the air). In another example, the alert may indicate that a manufacturing process is in error because a desired chemical or substance has not been detected in the product.

FIG. 11 is a block diagram illustrating entities of an example machine able to read instructions from a machine-readable medium and execute those instructions in a processor to perform the machine processing tasks discussed herein, such as the engine operations discussed above. Specifically, FIG. 11 shows a diagrammatic representation of a machine in the example form of a computer system 1100 within which instructions 1124 (e.g., software) for causing the machine to perform any one or more of the methodologies discussed herein may be executed. In alternative embodiments, the machine operates as a standalone device or may be connected (e.g., networked) to other machines, for instance, via the Internet. In a networked deployment, the machine may operate in the capacity of a server machine or a client machine in a server-client network environment, or as a peer machine in a peer-to-peer (or distributed) network environment.

The machine may be a server computer, a client computer, a personal computer (PC), a tablet PC, a set-top box (STB), a personal digital assistant (PDA), a cellular telephone, a smartphone, a web appliance, a network router, switch or bridge, or any machine capable of executing instructions 1124 (sequential or otherwise) that specify actions to be taken by that machine. Further, while only a single machine is illustrated, the term “machine” shall also be taken to include any collection of machines that individually or jointly execute instructions 1124 to perform any one or more of the methodologies discussed herein.

The example computer system 1100 includes a processor 1102 (e.g., a central processing unit (CPU), a graphics processing unit (GPU), a digital signal processor (DSP), one or more application-specific integrated circuits (ASICs), one or more radio-frequency integrated circuits (RFICs), or any combination of these), a main memory 1104, and a static memory 1106, which are configured to communicate with each other via a bus 1108. The computer system 1100 may further include a graphics display unit 1110 (e.g., a plasma display panel (PDP), a liquid crystal display (LCD), a projector, or a cathode ray tube (CRT)). The computer system 1100 may also include alphanumeric input device 1112 (e.g., a keyboard), a cursor control device 1114 (e.g., a mouse, a trackball, a joystick, a motion sensor, or other pointing instrument), a data store 1116, a signal generation device 1118 (e.g., a speaker), an audio input device 1126 (e.g., a microphone) and a network interface device 1120, which also are configured to communicate via the bus 1108.

The data store 1116 includes a machine-readable medium 1122 on which is stored instructions 1124 (e.g., software) embodying any one or more of the methodologies or functions described herein. The instructions 1124 (e.g., software) may also reside, completely or at least partially, within the main memory 1104 or within the processor 1102 (e.g., within a processor's cache memory) during execution thereof by the computer system 1100, the main memory 1104 and the processor 1102 also constituting machine-readable media. The instructions 1124 (e.g., software) may be transmitted or received over a network (not shown) via network interface 1120.

While machine-readable medium 1122 is shown in an example embodiment to be a single medium, the term “machine-readable medium” should be taken to include a single medium or multiple media (e.g., a centralized or distributed database, or associated caches and servers) able to store instructions (e.g., instructions 1124). The term “machine-readable medium” shall also be taken to include any medium that is capable of storing instructions (e.g., instructions 1124) for execution by the machine and that cause the machine to perform any one or more of the methodologies disclosed herein. The term “machine-readable medium” includes, but should not be limited to, data repositories in the form of solid-state memories, optical media, and magnetic media.

In this description, the term “module” refers to computational logic for providing the specified functionality. A module can be implemented in hardware, firmware, and/or software. Where the modules described herein are implemented as software, the module can be implemented as a standalone program, but can also be implemented through other means, for example as part of a larger program, as any number of separate programs, or as one or more statically or dynamically linked libraries. It will be understood that the named modules described herein represent one embodiment, and other embodiments may include other modules. In addition, other embodiments may lack modules described herein and/or distribute the described functionality among the modules in a different manner. Additionally, the functionalities attributed to more than one module can be incorporated into a single module. In an embodiment where the modules as implemented by software, they are stored on a computer readable persistent storage device (e.g., hard disk), loaded into the memory, and executed by one or more processors as described above in connection with FIG. 11. Alternatively, hardware or software modules may be stored elsewhere within a computing system.

As referenced herein, a computer or computing system includes hardware elements used for the operations described here regardless of specific reference in FIG. 11 to such elements, including, for example, one or more processors, high-speed memory, hard disk storage and backup, network interfaces and protocols, input devices for data entry, and output devices for display, printing, or other presentations of data. Numerous variations from the system architecture specified herein are possible. The entities of such systems and their respective functionalities can be combined or redistributed.

1. A non-transitory computer-readable medium comprising executable instructions, the executable instructions being executable by one or more processors to perform a method, the method comprising:
receiving a first set of intensity values, the first set of intensity values being based on a first set of intensity measurements for a set of wavelengths, the first set of intensity measurements obtained by an apparatus configured to generate first light, detect the first light that has passed through at least a first portion of a sample, and measure intensity of the first light by at least one optical sensor, a first angle separating the at least one optical sensor and the apparatus configured to generate light;
applying a first trained model to the first set of intensity values to obtain a first result;
based on the first result, determining a first subset of wavelengths of the first set of wavelengths;
receiving a second set of intensity values, the second set of intensity values being based on a second set of intensity measurements for the set of wavelengths, the second set of intensity measurements obtained by the apparatus configured to generate second light, detect the second light that has passed through at least a second portion of the sample, and measure intensity of the second light by the at least one optical sensor, a second angle separating the at least one optical sensor and the apparatus configured to generate light, the first angle being different than the second angle;
applying a second trained model to the second set of intensity values to obtain a second result, the first and second trained model being different from each other;
applying the first and second result to a trained multi-model to determine either a positive pathogen detection or a negative pathogen detection for the pathogen in the sample;
generating a pathogen detection notification that indicates either the positive pathogen detection or the negative pathogen detection for the pathogen in the sample; and
providing the pathogen detection notification.

2. The non-transitory computer-readable medium of claim 1, the method further comprising:
based on the output from the trained multi-model, determining a confidence level of the positive pathogen detection or the negative pathogen detection of the pathogen in the sample.

3. The non-transitory computer-readable medium of claim 1, the method further comprising based on the output from the trained multi-model, determining a distribution of pathogen particle size in the sample.

4. The non-transitory computer-readable medium of claim 1, the method further comprising based on the output from the trained multi-model, determining a concentration of the pathogen in the sample.

5. The non-transitory computer-readable medium of claim 1, wherein the apparatus includes at least one incoherent light source.

6. The non-transitory computer-readable medium of claim 1, wherein the apparatus includes a plurality of light emitting diodes (LEDs) and wherein the first angle separating at least one optical sensor and the apparatus utilizes one of the plurality of LEDs and the second angle separating at least one optical sensor and the apparatus utilizes another of the plurality of LEDs.

7. The non-transitory computer-readable medium of claim 1, wherein the at least one optical sensor includes at least two optical sensors and wherein the first angle separating at least one optical sensor and the apparatus utilizes a first optical sensor and the second angle separating at least one optical sensor and the apparatus is achieve utilizes a second optical sensor.

8. The non-transitory computer-readable medium of claim 1, wherein the first angle separating the at least one optical sensor and the apparatus configured to generate light is substantially 180 degrees.

9. The non-transitory computer-readable medium of claim 1, wherein the first angle and the second angle are substantially the same and wherein the first set of intensity measurements is obtained by positioning the sample in a first position and the second set of intensity measurements is obtained by positioning the sample in a second position, the first position being different from the second position.

10. The non-transitory computer-readable medium of claim 1, the method further comprising:
receiving a third set of intensity values, the third set of values based on a third set of intensity measurements for the set of wavelengths, the third set of intensity measurements obtained by the apparatus configured to generate light, detect the light that has passed through at least the portion of the sample, and measure intensity of light at least one optical sensor, a third angle separating the at least one optical sensor and the apparatus configured to generate light; and
applying a third trained model to the third set of intensity values to obtain a third result, wherein the determining either the positive pathogen detection or the negative pathogen detection for the pathogen in the sample is based on the output of the multi-model from the input of the first, second, and third results.

11. The non-transitory computer-readable medium of claim 1, wherein at least some training samples of the set of training samples correspond to a particular food processing facility, a region that includes multiple food processing facilities, or one or more classes of food processing facilities.

12. The non-transitory computer-readable medium of claim 1, wherein the result indicates a positive foodborne pathogen detection if the result meets or exceeds a threshold.

13. The non-transitory computer-readable medium of claim 1, wherein the set of wavelengths of light includes wavelengths of light in ultraviolet, visible, and infrared spectrums.

14. The non-transitory computer-readable medium of claim 1, wherein the second set of intensity values is based on scattered radiation from a source, the second trained model being a trained to analyze scattered radiation and the first trained model being trained to analyze absorbed radiation.

15. The non-transitory computer-readable medium of claim 1, wherein the at least one optical sensor comprises a first optical sensor that measures the intensity of the first light and a second optical sensor that measures the intensity of the second light.

16. The non-transitory computer-readable medium of claim 1, wherein the first trained model includes a first decision tree and the second trained model includes a second decision tree.

17. A system comprising at least one processor and memory containing executable instructions, the executable instructions being executable by the at least one processor to:
receive a first set of intensity values, the first set of intensity values being based on a first set of intensity measurements for a set of wavelengths, the first set of intensity measurements obtained by an apparatus configured to generate first light, detect the first light that has passed through at least a first portion of a sample, and measure intensity of the first light by at least one optical sensor, a first angle separating the at least one optical sensor and the apparatus configured to generate light;
apply a first trained model to the first set of intensity values to obtain a first result;
based on the first result, determine a first subset of wavelengths of the first set of wavelengths;
receive a second set of intensity values, the second set of intensity values being based on a second set of intensity measurements for the set of wavelengths, the second set of intensity measurements obtained by the apparatus configured to generate second light, detect the second light that has passed through at least a second portion of the sample, and measure intensity of the second light by the at least one optical sensor, a second angle separating the at least one optical sensor and the apparatus configured to generate light, the first angle being different than the second angle;
apply a second trained model to the second set of intensity values to obtain a second result, the first and second trained model being different from each other;
applying the first and second result to a trained multi-model to determine either a positive pathogen detection or a negative pathogen detection for the pathogen in the sample;
generate a pathogen detection notification that indicates either the positive pathogen detection or the negative pathogen detection for the pathogen in the sample; and
provide the pathogen detection notification.

18. The system of claim 17, wherein the executable instructions being executable by the at least one processor to determine a confidence level of the positive pathogen detection or the negative pathogen detection of the pathogen in the sample, based on the output from the trained multi-model.

19. The system of claim 17, wherein the executable instructions being executable by the at least one processor to generate the second set of values based on the first set of values include executable instructions being executable by the at least one processor to determine a distribution of pathogen particle size in the sample based on the output from the trained multi-model.

20. The system of claim 17, wherein the executable instructions being executable by the at least one processor to determine a concentration of the pathogen in the sample based on the output from the trained multi-model.

21. The system of claim 17, wherein the apparatus includes at least one incoherent light source.

22. The system of claim 17, wherein the apparatus includes a plurality of light emitting diodes (LEDs) and wherein the first angle separating at least one optical sensor and the apparatus utilizes one of the plurality of LEDs and the second angle separating at least one optical sensor and the apparatus utilizes another of the plurality of LEDs.

23. The system of claim 17, wherein the at least one optical sensor includes at least two optical sensors and wherein the first angle separating at least one optical sensor and the apparatus utilizes a first optical sensor and the second angle separating at least one optical sensor and the apparatus is achieve utilizes a second optical sensor.

24. The system of claim 17, wherein the first angle separating the at least one optical sensor and the apparatus configured to generate light is substantially 180 degrees.

25. The system of claim 17, wherein the first angle and the second angle are substantially the same and wherein the first set of intensity measurements is obtained by positioning the sample in a first position and the second set of intensity measurements is obtained by positioning the sample in a second position, the first position being different from the second position.

26. The system of claim 17, wherein the executable instructions being executable by the at least one processor to:
receive a third set of intensity values, the third set of values based on a third set of intensity measurements for the set of wavelengths, the third set of intensity measurements obtained by the apparatus configured to generate light, detect the light that has passed through at least the portion of the sample, and measure intensity of light at least one optical sensor, a third angle separating the at least one optical sensor and the apparatus configured to generate light; and
apply a third trained model to the third set of intensity values to obtain a third result, wherein the determining either the positive pathogen detection or the negative pathogen detection for the pathogen in the sample is based on the output of the multi-model from the input of the first, second, and third results.

27. The system of claim 17, wherein at least some training samples of the set of training samples correspond to a particular food processing facility, a region that includes multiple food processing facilities, or one or more classes of food processing facilities.

28. The system of claim 17, wherein the result indicates a positive foodborne pathogen detection if the result meets or exceeds a threshold.

29. The system of claim 17, wherein the set of wavelengths of light includes wavelengths of light in ultraviolet, visible, and infrared spectrums.

30. A method comprising:
receiving a first set of intensity values, the first set of intensity values being based on a first set of intensity measurements for a set of wavelengths, the first set of intensity measurements obtained by an apparatus configured to generate first light, detect the first light that has passed through at least a first portion of a sample, and measure intensity of the first light by at least one optical sensor, a first angle separating the at least one optical sensor and the apparatus configured to generate light;
applying a first trained model to the first set of intensity values to obtain a first result;
based on the first result, determining a first subset of wavelengths of the first set of wavelengths;
receiving a second set of intensity values, the second set of intensity values being based on a second set of intensity measurements for the set of wavelengths, the second set of intensity measurements obtained by the apparatus configured to generate second light, detect the second light that has passed through at least a second portion of the sample, and measure intensity of the second light by the at least one optical sensor, a second angle separating the at least one optical sensor and the apparatus configured to generate light, the first angle being different than the second angle;
applying a second trained model to the second set of intensity values to obtain a second result, the first and second trained model being different from each other;
applying the first and second result to a trained multi-model to determine either a positive pathogen detection or a negative pathogen detection for the pathogen in the sample;
generating a pathogen detection notification that indicates either the positive pathogen detection or the negative pathogen detection for the pathogen in the sample; and
providing the pathogen detection notification.
